(19) United States
(12) Patent Application Publication
(10) Pub. No.: US 2012/0149046 A1
(43) Pub. Date: Jun. 14, 2012

(54) Bioluminescent Bacterial Detection

(76) Inventors: James Paul Meighan, Watford (GB); Frederic Nason, Camarillo, CA (US)

(21) Appl. No.: 13/390,981

(22) PCT Filed: Aug. 20, 2010
(86) PCT No.: PCT/GB2010/001578

§ 371 Date: Feb. 17, 2012

Related U.S. Application Data
(60) Provisional application No. 61/235,552, filed on Aug. 20, 2009.

(51) Int. Cl. G01N 21/76; C12M 1/40

(52) U.S. Cl. 435/8; 435/34; 435/18; 435/38; 435/19; 435/21; 435/23; 435/288.7

(57) ABSTRACT

This invention relates to bioluminescent methods for detecting specific target bacteria, such as E. coli, coliforms, Enterococcus spp, Listeria spp and S. aureus in samples. The sample to be tested is incubated in a non-selective growth medium for up to 8 hours to produce a sample culture and which is then mixed with detection reagents. The detection reagents include a lysis reagent which disrupts bacterial cells in the sample, a pro-luciferin molecule which is specifically converted into luciferin by said target bacteria; and luminescence reagents which produce a luminescent signal in the presence of luciferin. The mixture of sample culture and detection reagents is then incubated and the luminescent signal from the reaction mixture measured.

This invention relates to the specific detection of target bacteria in samples.

Various techniques are currently used for the detection of specific types of bacteria in samples.

Chromogenic agars contain chromogenic substrates which are cleaved by enzymes produced by the target bacteria. Cleavage of the substrate changes the colour of the colony or surrounding agar and indicates that the target bacteria are present. Examples of chromogenic agars include Brilliance™ (Oxoid) and CHROMagar™ (BioMerieux).

Chromogenic broths also contain chromogenic substrates which are cleaved by enzymes produced by the target bacteria. Cleavage of the substrate changes the colour of the broth and indicates that the target bacteria are present. Examples of chromogenic broths include Readycult™ (Merck KGaA) and Colilert™ (IDEXX).

Lateral flow methods employ a selection of antibodies to the target organism which are immobilised on a solid phase lateral flow strip. Binding to the antibodies is determined after an incubation period. Although for some lateral flow assays, the incubation period has been stated to be 8 hours, in reality an 18 hour incubation (i.e. overnight) is generally required. Examples of lateral flow detection assays include the SinglePath™ system (Merck KGaA) and the Lateral Flow System™ (DuPont).

Although these methods are employed across many different industrial sectors, they all require sample incubation for at least 18 hours to allow the resultant colour to develop before detection.

Molecular methods may also be used for the detection of specific types of bacteria in samples. For example, methods based on the polymerase chain reaction (PCR) are widely used. These techniques involve the amplification of bacterial DNA using complementary primers and various kits are commercially available. However, PCR is unable to distinguish between viable and dead or non-viable cells. PCR-based methods therefore commonly employ an incubation period to enrich the sample for viable cells.

The present invention relates to the development of bioluminescent techniques that detect specific target bacteria in samples with an unexpected combination of both speed and sensitivity.

One aspect of the invention provides a method of detecting target bacteria in a sample comprising;
a) providing a sample to be tested for target bacteria,
b) incubating the sample in non-selective growth medium to produce a sample culture,
c) mixing some or all of the sample culture with detection reagents to produce a reaction mixture;
wherein said detection reagents comprise;
   (i) a lysis reagent which disrupts bacterial cells in the sample,
   (ii) a pro-luciferin molecule which is specifically converted into luciferin by said target bacteria; and,
   (iii) luminescence reagents which produce a luminescent signal in the presence of luciferin;
d) incubating the reaction mixture, and;
e) measuring the luminescent signal from the reaction mixture following said incubation.

The detection of a luminescent signal from the reaction mixture may be indicative of the presence or amount of target bacteria in the sample.

Any suitable sample may be tested for target bacteria using the methods described herein.

In some embodiments, the sample may be a liquid sample, for example water, or a foodstuff, beverage, raw material, personal care product, bodily fluid, such as blood or urine, or medicinal formulation. A sample of a liquid to be tested for target bacteria may be removed using a dipper, pipette or other liquid sampler. Alternatively, the liquid may be passed through a filter, and material trapped on the filter recovered for testing for target bacteria. Suitable samplers and techniques for obtaining liquid samples for testing are well-known in the art.

In other embodiments, the sample may be from a surface, for example a hard surface, such as a surface for the preparation or service of food. A sample may be collected from a surface using a swab or other surface sampler. The swab may be moistened before use with a neutral wetting agent, such as MRD, or Butterfield's solution. Typically, an area of 100 cm² will be swabbed. The sampling of surfaces, for example for hygiene monitoring purposes, is well-known in the art.

In other embodiments, the sample may be a gaseous sample, for example an air sample. A sample of a gas to be tested for target bacteria may be obtained by passing the gas through a filter and recovering the material trapped on the filter for testing.

The sample is inoculated into the non-selective medium using standard techniques. The non-selective medium may be a solid or liquid medium. Typically, a sampler, such as a swab or dipper, containing the sample is immersed in non-selective growth medium in a culture vessel or device. In some embodiments, a sampler containing the sample is placed within a sample culture device, and a breakable barrier disrupted to immerse the sampler and the sample in non-selective growth medium. Suitable devices are described in more detail below. In other embodiments, the non-selective medium may be a solid medium. The sample may be plated onto the surface of the medium using standard microbiological techniques.

After inoculation, the non-selective growth medium containing the sample is incubated to produce the sample culture. Incubation is carried out under conditions suitable for bacterial growth. Suitable conditions are well-known in the art and typically include incubation at about 37° C., optionally with agitation or shaking of liquid media.

Incubation of the sample in the non-selective medium for a period of 8 hours or less increases the specificity of the detection and reduces the occurrence of false positives, since the low-level conversion of the pro-luciferin molecule into luciferin by non-target bacteria does not reach a threshold value within the incubation period that would be taken as a positive signal.

The sample may be incubated in the non-selective growth medium for 8 hours or less, 7 hours or less, 6 hours or less, 5 hours or less, 4 hours or less, 3 hours or less, 2 hours or less or 1 hour or less to produce the sample culture.

The length of the sample incubation, along with the target bacteria and the culture conditions, affects the statistical probability of detecting certain numbers of target bacteria. The probability of detection for any particular set of test conditions may be determined using standard techniques.

For example, an initial probability table may be derived from inoculations of known numbers of target bacterial cells. Plots of the distribution of data for the detection of target bacterial cells at each inoculation level in replicate control samples may be used to estimate the probability of detection for a particular set of test conditions.

A table of probability of detection of coliforms is shown in Table 8. For example, the probabilities set out in Table 8 show that 8 hour incubation provides for 95% confidence of detecting a single coliform cell. In other words, if 1 cell exists in the medium at time of initial inoculation then 95 times out of 100, that single cell will produce a detectable positive signal after 8 hours incubation.

For any particular test, the incubation time will be determined by the sensitivity and confidence limits which are required.

A non-selective medium is a nutritious medium capable of supporting the uninhibited and unlimited growth of culturable bacteria without limits to the growth. Non-selective media are devoid of antibiotics or other selective agents and contain all the nutrients required to support the growth of all culturable bacteria. Suitable non-selective liquid growth medium are well-known in the art and include Tryptone Soya Broth, Nutrient Broth and Brain Heart Infusion Broth. Corresponding non-selective solid growth medium may be produced, for example, by the addition of agar (e.g. 1.5% w/v). Suitable non-selective bacteria growth medium may be obtained from commercial suppliers (e.g. Oxoid, Fluka, Sigma-Aldrich).

In some embodiments, for example when the sample is a water sample, solid non-selective growth medium may be preferred. Solid growth medium inoculated with sample may be incubated for 1 hour or less to produce a sample culture which comprises individual or confluent colonies growing on the surface of the medium. Following incubation, one or more colonies from the sample culture may be removed and mixed with the detection reagents to produce the reaction mixture.

The non-selective medium is generally free of viable micro-organisms before inoculation with the sample (i.e. sterile). In some preferred embodiments, the non-selective medium is sterilised by a method other than autoclaving, for example filtration, to reduce the bioluminescent background signal.

In some embodiments, the non-selective growth medium may further comprise a compound which increases the conversion of the pro-luciferin molecule into luciferin by the target bacteria, for example by inducing the expression of the enzyme which is detected. For example, the non-selective liquid growth medium may be supplemented with isopropylthiogalactoside (IPTG) to induce β-galactosidase expression and facilitate the detection of coliforms; methyl-β-glucuronide to induce β-glucuronidase expression and facilitate the detection of E. coli; glycerol to induce β-glucosidase expression and facilitate the detection of Enterococcus spp; or NaCl to induce PiPL and facilitate the detection of pathogenic Listeria spp.

In some embodiments, where large amounts of bacteria are expected, a bacterial growth inhibitor, preferably a non-selective bacterial growth inhibitor, such as sodium azide, may be added to the non-selective liquid medium; for example to detect Enterococcus spp in seawater.

In embodiments in which the sample contains large amounts of bacteria, it may be used directly as a sample culture without incubation in non-selective medium. For example, a sample of urine from a patient with a UTI (Urinary Tract Infection) may contain more than 1,000,000 cells/ml and may be used as the sample culture in the methods described herein to directly detect the bacteria therein.

A method of detecting target bacteria in a sample comprising;
a) providing a sample to be tested for target bacteria,
b) mixing some or all of the sample with detection reagents to produce a reaction mixture;
wherein said detection reagents comprise;
   (i) a lysis reagent which disrupts bacterial cells in the sample,
   (ii) a pro-luciferin molecule which is specifically converted into luciferin by said target bacteria; and,
   (iii) luminescence reagents which produce a luminescent signal in the presence of luciferin;
c) incubating the reaction mixture, and;
d) measuring the luminescent signal from the reaction mixture following said incubation.

Suitable samples include urine samples, for example urine samples from individuals suspected of having a UTI. Suitable target bacteria include coliforms and E. coli, which may be detected as described herein.

After incubating the sample in non-selective medium to produce a sample culture, or alternatively, taking the sample culture directly from the sample, as described above, the sample culture is tested for the target bacteria.

In some embodiments, the sensitivity of the method may be increased before testing for the target bacteria by centrifugation of a volume of the sample culture. The cellular pellet may be resuspended in a reduced volume of medium or buffer, such as 100 mM Tris: BES pH 8.00 or 100 mM Tris: BES pH 8.00 (i.e. less than the centrifuged volume of sample culture), before being contacted with the detection reagents. For example, the sample culture may be centrifuged at 5000 RPM for 5 minutes. This increases the concentration of cells in the sample culture prior to contact with the detection reagents, thereby further increasing the sensitivity of the assay.

The sample culture is tested for the target bacteria by admixing some or all of the sample culture with detection reagents which comprise a pro-luciferin molecule.

The detection reagents may be added to the sample culture or a colony, portion or aliquot thereof in a single solution or may be added in two or more separate solutions.

For example, in some embodiments, a buffer solution may be added to the sample culture or portion thereof initially or a colony of the sample culture may be suspended in a buffer solution, and the pro-luciferin molecule and luminescence reagents may then be added. The lysis reagent may be added with either the buffer solution or the luminescence reagents.

In some embodiments, the sample culture may be separately tested for the presence of 2, 3, 4, 5, 6 or more different target bacteria. The results of the separate tests may be used to produce a profile of the bacteria in the sample, which may facilitate identification and characterisation. For example, the sample culture may be separately tested for E. coli, coliforms, Listeria spp, Enterococcus spp, protease-producing species and phosphatase-producing species as described herein. Examples of profiles based on these tests are shown in Table 13.

The lysis reagent disrupts bacterial cells in the sample culture and releases intracellular enzymes into the medium. In the presence of target bacteria, the amount of the characteristic bacterial enzyme which is exposed to pro-luciferin molecule is increased by the lysis reagent, and therefore the production of the luminescent signal is increased.

Suitable lysis reagents disrupt bacterial cells but do not substantially inhibit enzymatic reactions, such as the conversion of pro-luciferin to luciferin or the luciferase-mediated production of bioluminescence. Suitable lysis reagents include chlorohexidine digluconate (CHDG), NRM™ reagent (Hygiena Int, CA), quaternary ammonium compounds, such as benethonium chloride, and quaternary ammonium derivatives. A suitable buffer solution may have a pH 7 to pH 9, preferably pH 8. For example 100 mM Tris: BES pH 8.0 may be employed.

In some embodiments, the lysis reagent may be omitted from the detection reagents and the target bacteria may be detected without disruption of the bacterial cells in the culture sample.

A pro-luciferin molecule is an inactive luciferin precursor which is not a luciferase substrate but is converted into luciferin by the target bacteria. The term luciferin includes firefly luciferin ((4S)-2-(6-hydroxy-1,3-benzothiazol-2-yl)-4,5-dihydrothiazole-4-carboxylic acid) and luciferin derivatives, such as aminoluciferin, which are substrates for luciferase. The target bacteria produce an enzyme which is characteristic of the target bacteria i.e. the enzyme is produced by target bacteria and not by other bacteria or the enzyme is produced at higher levels by target bacteria than by other bacteria. The characteristic bacterial enzyme catalyses the conversion of pro-luciferin into luciferin in the presence of the target bacteria. For example, a pro-luciferin molecule may comprise a luciferin moiety and a blocking moiety that prevents the luciferin moiety from reacting with luciferase. The choice of blocking moiety and type of linkage to the luciferin moiety depends on the characteristic bacterial enzyme. Suitable linkages may for example include ether, glycosyl or peptidyl linkages. The characteristic bacterial enzyme may be intracellular and only exposed to the pro-luciferin molecule when the cell is disrupted, or a secreted enzyme and exposed to the pro-luciferin molecule before the cell is disrupted. Luciferin which is produced from the pro-luciferin molecule by the characteristic bacterial enzyme is a luciferase substrate and is converted by the luminescence reagents, which include luciferase and ATP, into oxyluciferin, with the concomitant production of light.

In alternative aspects of the invention, the pro-luciferin molecule may be replaced by a pro-coelenterazine molecule. For all the methods described herein which employ a pro-luciferin molecule, the invention also provides the corresponding methods employing a pro-coelenterazine molecule instead of the pro-luciferin molecule. Coelenterazine which is produced from the pro-luciferin molecule by the characteristic bacterial enzyme is a Renilla luciferase substrate and is converted by the luminescence reagents, which include Renilla luciferase and ATP, into oxycoelenterazine, with the concomitant production of light.

Examples of characteristic bacterial enzymes include β-galactosidase, which is characteristic of coliforms, β-glucoronidase, which is characteristic of E. coli, β-lactamase, which is characteristic of extended spectrum β-lactamase (ESBL) organisms, β-glucosidase, which is characteristic of Enterococcus spp, Yersinia spp, and Listeria spp, phospholipase C, which is characteristic of pathogenic Listeria spp, ribonuclease, which is characteristic of Salmonella spp, alkaline phosphatase, which is characteristic of S. aureus, cytochrome oxidase which is characteristic of Pseudomonas spp and Vibrio spp, non-specific protease, which is characteristic of a class of protease producing bacteria, and α-glucosidase and β-cellobiosidase, which are characteristic of Enterobacter sakazakii.

In some embodiments, the characteristic bacterial enzyme is secreted by the target bacteria into the medium. This may be sufficient to produce a luminescent signal in the presence of pro-luciferin molecules and luminescence reagents, without disrupting the bacterial cells in the culture.

The target bacteria may be a specific strain, species, genus or any other group or class of bacteria whose members express the characteristic bacterial enzyme and therefore share the ability to convert the pro-luciferin molecule into luciferin. Different pro-luciferin molecules may be used to identify target bacteria at different taxonomic levels, as required by the operator. This may be helpful for example in characterising the bacterial enzymes expressed by bacteria in the sample for example to produce a biochemical profile.

The target bacteria may be coliforms. Coliforms include lactose positive Enterobacteriacae, such as E. coli, Citrobacter spp, Enterobacter spp, and Klebsiella spp. β-galactosidase is a characteristic enzyme expressed by coliforms. Methods for the detection of coliforms as described herein may employ pro-luciferin molecules which are converted by β-galactosidase activity into luciferin. For example, a suitable pro-luciferin molecule may comprise a luciferin moiety and a β-galactoside moiety. Pro-luciferin molecules which might be used in the detection of coliforms include luciferin-O-β-galactoside or luciferin-O-β-D-galacto-pyranoside.

The target bacteria may be E. coli. β-glucoronidase is a characteristic enzyme expressed by E. coli. Methods for the detection of E. coli as described herein may employ pro-luciferin molecules which are converted by β-glucoronidase activity into luciferin. For example, a suitable pro-luciferin molecule may comprise a luciferin moiety and a β-glucuronide moiety. Pro-luciferin molecules which might be used in the detection of E. coli include luciferin-O-β-glucuronide.

The target bacteria may be an ESBL organism. ESBL organisms are Enterobacteriaceae or coliforms which express β-lactamase enzymes. These beta lactamases confer antibiotic resistance. Methods for the detection of ESBL organisms as described herein may employ pro-luciferin molecules which are converted by beta-lactamase activity into luciferin. For example, a suitable pro-luciferin molecule may comprise a mono-lactam-moiety and a luciferin moiety connected via a linkage which is cleaved by β-lactamase. Pro-luciferin molecules which might be used in the detection of ESBL organisms include β-lactamyl-luciferins, for example cephalosporin-linked luciferins, such as cephalosporin-O-β-luciferin and penicillin-linked luciferins, such as penicillin-O-β-luciferin.

The target bacteria may be Enterobacter sakazakii. α-Glucosidase and β-cellobiosidase are characteristically expressed by Enterobacter sakazakii. Methods for the detection of Enterobacter sakazakii as described herein may employ pro-luciferin molecules which are converted by α-glucosidase activity into luciferin and pro-luciferin molecules which are converted by β-cellobiosidase activity into luciferin. For example, a suitable pro-luciferin molecule may comprise a luciferin moiety and an α-glucose moiety or a β-cellobiose moiety. Pro-luciferin molecules which might be used in the detection of Enterobacter sakazakii include luciferin-α-glucoside and luciferin-β-cellobiose.

In some embodiments, either an α-glucosidase or a β-cellobiosidase labile pro-luciferin is used to detect Enterobacter sakazakii. The other activity may be detected as a confirmation, for example using chromogenic substrate, as described herein. In other embodiments, a bi-functional pro-luciferin molecule comprising a luciferin moiety; a α-glucose moiety; and a p-cellobiose moiety may be employed. This substrate is converted to luciferin only in the presence of both α-glucosidase and β-cellobiosidase. Bifunctional substrates are described in more detail below.

The target bacteria may be Enterococcus spp. β-glucosidase is a characteristic enzyme expressed by Enterococcus spp. Methods for the detection of Enterococcus spp as described herein may employ pro-luciferin molecules which are converted by β-glucosidase activity into luciferin. For example, a suitable pro-luciferin molecule may comprise a luciferin moiety and a beta-glucoside moiety. Pro-luciferin molecules which might be used in the detection of Enterococcus spp include luciferin-O-β-glucoside or luciferin-O-β-D-gluco-pyranoside.

The target bacteria may be Yersinia spp. β-glucosidase is a characteristic enzyme expressed by Yersinia spp. Methods for the detection of Yersinia spp as described herein may employ pro-luciferin molecules which are converted by β-glucosidase activity into luciferin. For example, a suitable pro-luciferin molecule may comprise a luciferin moiety and a beta-glucoside moiety. Pro-luciferin molecules which might be used in the detection of Yersinia spp include luciferin-O-β-glucoside or luciferin-O-β-D-gluco-pyranoside.

The target bacteria may be Listeria spp. β-glucosidase is a characteristic enzyme expressed by Listeria spp. Methods for the detection of Listeria spp as described herein may employ pro-luciferin molecules which are converted by β-glucosidase activity into luciferin. For example, a suitable pro-luciferin molecule may comprise a luciferin moiety and a beta-glucoside moiety. Pro-luciferin molecules which might be used in the detection of Listeria spp include luciferin-O-β-glucoside.

In order to differentiate Yersinia, Enterococcus and Listeria spp in a β-glucosidase assay, if required, an Enterococcal inhibitor, such as LiCL may be added to the non-selective liquid medium.

The target bacteria may be a pathogenic Listeria spp, such as L. monocytogenes and L. ivanovii. PCPLC (phosphatidylcholine phospholipase C) and PiPLC (phosphatidylinositol phospholipase C) are characteristically expressed by pathogenic Listeria spp. Methods for the detection of pathogenic Listeria spp as described herein may employ pro-luciferin molecules which are converted by PCPLC activity into luciferin and/or pro-luciferin molecules which are converted by PiPLC activity into luciferin. For example, a suitable pro-luciferin molecule may comprise a luciferin moiety and a phosphatidylcholine moiety, such as luciferin-o-phosphatidylcholine. Other suitable pro-luciferin molecules may comprise a luciferin moiety and a phosphotidylinositol moiety, such as luciferin-o-phosphatidylinositol or luciferin-o-myo-inositol-1-phosphate.

The target bacteria may be S. aureus. Alkaline phosphatase is characteristically expressed by S. aureus. Methods for the detection of S. aureus as described herein may employ pro-luciferin molecules which are converted by alkaline phosphatase activity into luciferin. For example, a suitable pro-luciferin molecule may comprise a luciferin moiety and a phosphate group. Pro-luciferin molecules which might be used in the detection of S. aureus include luciferin-O-phosphate and benzyl-luciferin-O-phosphate.

Methicillin resistant S. aureus (MRSA) may be distinguished from other strains of S. aureus by supplementing the non-selective medium with methicillin or a derivative thereof (e.g. oxicillin), or performing a confirmatory test with methicillin-supplemented medium.

The target bacteria may be a Salmonella spp. Deoxyribonuclease is characteristically expressed by Salmonella spp. Methods for the detection of Salmonella spp as described herein may employ pro-luciferin molecules which are converted by deoxyribonuclease activity into luciferin. For example, a suitable pro-luciferin molecule may comprise a luciferin moiety and a 2-deoxy-D-ribose group. Pro-luciferin molecules which might be used in the detection of Salmonella spp include 2-deoxy-D-ribosyl-luciferin.

Other enzymes which are characteristic of Salmonella spp and may be used for detection as described herein include α-galactosidase and fatty acid esterases such as octanoate esterase or nonanoate esterase. Methods for the detection of Salmonella spp as described herein may employ pro-luciferin molecules which are converted by α-galactosidase or a fatty esterase into luciferin. For example, a suitable pro-luciferin molecule may comprise a luciferin moiety and an α-galactoside group or a fatty acyl group. Pro-luciferin molecules which might be used in the detection of Salmonella spp include luciferin-O-α-galactoside, luciferin-O-octanoate and luciferin caprylate-nonanoate.

The target bacteria may be a cytochrome oxidase producing organism, for example, a Pseudomonas spp, Vibrio spp or an associated organism. Methods for the detection of cytochrome oxidase producing organisms as described herein may employ pro-luciferin molecules which are converted by cytochrome oxidase activity into luciferin. For example, a suitable pro-luciferin molecule may comprise a luciferin moiety and a blocking group connected through an ether linkage. Suitable pro-luciferin molecules are commercially available (e.g. Luciferin-MultiCYP, Luciferin-2J2/4F12, Luciferin-4F12, Luciferin-4F2/3, Luciferin-4A11 and Luciferin-3A7; all from Promega Inc, WI USA).

The target bacteria may be a protease producing organism, for example, E. coli 0157 and certain Staphylococcus spp and Salmonella spp. Methods for the detection of protease producing organisms as described herein may employ pro-luciferin molecules which are converted by a non-specific protease activity into luciferin. For example, a suitable pro-luciferin molecule may comprise a luciferin moiety and a peptidyl group, typically 1, 2, 3, 4 or more amino acids. A peptidyl group may be conveniently attached to aminoluciferin via a peptide bond. Pro-luciferin molecules may be based on known trypsin and carboxypeptidase substrates. Suitable molecules are available commercially and include AAF-luciferin, LRR-luciferin, GP-luciferin, DEVD-luciferin, LETD-luciferin, LEHD-luciferin and VDVAD-luciferin (Promega Inc WI USA).

In some embodiments, the pro-luciferin molecule may be converted into luciferin by the action of two or more bacterial enzymes. For example, a pro-luciferin molecule may comprise a luciferin moiety linked to first and second inhibitor moieties. A first bacterial enzyme may remove the first inhibitor moiety and a second bacterial enzyme may remove the second inhibitor moiety. Only in the presence of both the first and second bacterial enzymes is the active luciferin produced and a luminescent signal generated. In other embodiments, the pro-luciferin molecule may be converted into luciferin by the action of a bacterial enzyme and another enzyme present in the detection reagents. Only in the presence of both the bacterial enzyme and the detection reagents is the active luciferin produced and a luminescent signal generated.

Bifunctional pro-luciferin molecules may be useful in increasing the specificity of detection, for example in the differentiation of closely related species of bacteria. For example, a pro-luciferin molecule which comprises both β-cellobioside and α-glucoside moieties may be useful in distinguishing Enterobacter sakazakii from other Enterobacter species. A pro-luciferin molecule which comprises both β-galactoside and β-glucuronide moieties may be useful in distinguishing E. coli from Shigella spp.

A pro-luciferin molecule is an inactive luciferin precursor which is not a luciferase substrate but is converted into luciferin by the target bacteria. The target bacteria produce an enzyme which is characteristic of the target bacteria i.e. the enzyme is produced by target bacteria and not by other bacteria or the enzyme is produced at higher levels by target bacteria than by other bacteria. The characteristic bacterial enzyme, optionally in combination with a second enzyme from bacteria or the detection reagents, catalyses the conversion of pro-luciferin into luciferin in the presence of the target bacteria.

Similarly, pro-coelenterazine molecules may be used in the methods described herein mutatis mutandis instead of pro-luciferin molecules. A pro-coelenterazine molecule is an inactive coelenterazine precursor which is not a Renilla luciferase substrate but is converted into coelenterazine by the target bacteria. The characteristic bacterial enzyme, optionally in combination with a second enzyme from bacteria or the detection reagents, catalyses the conversion of pro-coelenterazine into coelenterazine in the presence of the target bacteria.

Suitable pro-luciferin molecules and pro-coelenterazine molecules for use in the present methods may be produced using standard techniques of chemical synthesis. After synthesis, a pro-luciferin molecule may be purified to reduce the background signal produced in assays. Suitable purification techniques include HPLC, for example reverse phase HPLC. Suitable pro-luciferin molecules and pro-coelenterazine molecules may also be available from commercial sources (e.g. Promega Inc, USA).

Preferably, the pro-luciferin molecule is at least 99.9% pure.

A sample of pro-luciferin molecule may be further treated with luciferase to convert any contaminating luciferin into oxyluciferin and thereby reduce the background signal.

The concentration of pro-luciferin molecule in the detection reagents is generally high enough to be non-rate limiting on the production of a bioluminescent signal when contacted with the sample culture, without producing a significant background signal in the absence of target bacteria. For example, the detection reagents may comprise 1 to 100 μg/ml, preferably 10 μg/ml of the pro-luciferin molecule. The overall concentration of the pro-luciferin molecule in the culture following addition of the detection reagents is preferably from 1 to 10 μg/ml, for example about 3 μg/ml.

The detection reagent further comprises luminescence reagents.

Luminescence reagents are reagents which, in combination, lead to the production of a bioluminescent signal in the presence of luciferin. Suitable luminescence reagents are well-known in the art. For example, luminescent reagents may comprise luciferase, ATP, Mg2+, a reducing agent, such as dithiothreitol (DTT) and a chelating agent, such as EDTA. Additional stabilisers may be added, if required.

Suitable luciferases include firefly luciferase (for pro-luciferin substrates) and renilla luciferase (for pro-coelenterazine substrates) and are available commercially (e.g. Sigma Aldrich, Promega Corp). Other luciferases are also available in the art and may be used as described herein. The amount of luciferase and ATP is generally high enough to be non-rate limiting on the production of a bioluminescent signal. Typically, the final concentration of luciferase following addition of detection reagents to the sample culture may be 5 to 10 μg/ml, preferably about 6 μg/ml.

The final concentration of ATP in the reaction mixture is 1 μM to 10 mM, preferably 1 μM to 1 mM.

The final concentration of Mg²⁺ in the reaction mixture is 1 mM to 100 mM, preferably about 10 mM.

The final concentration of DTT in the reaction mixture is 100 μM to 10 mM, preferably about 1 mM.

The final concentration of EDTA in the reaction mixture is 500 μM to 50 mM, preferably about 5 mM.

Following addition of the detection reagents to the sample culture, the luminescent signal produced by the reaction mixture may be measured after a fixed time period (known as the “assay time”). For example, the signal may be measured at least 5 mins, at least 10 mins, or at least 15 mins after the detection reagents are added to the sample culture. In some embodiments, the signal may be measured up to 60, 75 or 90 mins after addition of the detection reagents.

Preferably, the luminescent signal is measured after 10 to 60 mins at 37° C., following addition of the detection reagents. Typical assay times include 5, 10, 15, 20, 25, 30 or 40 mins. A suitable assay time for a particular set of test conditions may be readily determined using standard techniques, in order to achieve the required probability of detection. For optimal sensitivity, long sample culture incubation times and long assay times may be employed. When short sample culture incubation times are used, a long assay time may be employed, for increased sensitivity. Where sensitivity is less important, for example, when short sample culture incubation times are used, a short assay time may be employed. Typically, a 2 hour or 4 hour sample culture incubation with a 20 min assay time may be employed, or a 4 hour sample culture incubation with a 10 min assay time.

The detection or measurement of a luminescent signal may be carried out using a luminometer in accordance with conventional techniques. Examples of commercially available luminometers include the Pi 102 PMT luminometer or SystemSure Plus™ (Hygiena Int, CA USA).

As described above, the luminescent signal from the reaction mixture may be measured at a single time point. This may be useful in determining whether or not the target bacteria are present in the sample. For example, the luminescent signal may be measured after a fixed incubation period e.g. 4, 6, or 8 hours, and compared to a predetermined threshold value. A signal above the threshold value indicates that the target bacterium is present in the sample.

The threshold value may be determined for any particular set of reagents, conditions and desired confidence limits by measuring the luminescent signal from control cultures e.g. medium without cells; cultures of non-target bacteria and cultures of known amounts of target bacteria under the test conditions.

The pre-determined threshold value may be a value which is significantly greater than the background signal (i.e. the signal obtained from controls in the absence of target bacteria). The greater the threshold value over the background signal, the greater the confidence that a positive signal indicates the presence of target bacteria in the sample. For example, a suitable threshold value may be determined to be at least the background signal+3 standard deviations.

A pre-determined threshold value may be validated using a series of control cultures of known amounts of target bacteria to determine if any of these control cultures are slower or quicker to produce a luminescent signal up to and beyond the pre-determined threshold value at set time intervals in the sample incubation.

In any particular test, the threshold level may be set according to the pass/fail criteria for the test and the confidence limits required. For example, the threshold level may be set to pass less than 1000 E. coli with 95% confidence limits. The threshold level may be set for particular pass/fail criteria and confidence limits from control curves produced using known amounts of target cells.

The amount of luminescent signal after incubation with the detection reagent may be indicative of the number of target bacteria in the sample. For example, a high luminescent signal may be indicative of high numbers of target bacteria in the sample. However, for determining the starting number of target bacteria in the original sample, the luminescent signal from the sample culture is preferably measured periodically i.e. at two or more time points during incubation. For example, the luminescent signal may be measured at two or more time points (e.g. any of 2 4, 6, and 8 hours), and compared to calibration data obtained from control cultures of target bacteria for a particular set of incubation and assay times, reagents and the luminometer used, to determine the numbers of cells in the culture sample at each time point. A growth curve may then be plotted and/or a growth rate equation derived. The amount of target bacteria in the original sample can then be calculated by extrapolating the growth curve or growth rate equation back to time zero.

The luminescent signal from the sample culture may be measured at two time points, for example 2 and 4 hours or 2 and 6 hours. This may be useful in providing a semi-quantitative determination of the number of target bacteria in the sample.

The luminescent signal from the sample culture may be measured at three or more time points, for example 2, 4 and 6 hours. This may be useful in providing a quantitative determination of the number of target bacteria in the sample.

The amount of target bacteria in the original sample may be determined using a predetermined algorithm based on the required confidence limits, incubation time, assay time and instrument used.

In some embodiments, the detection reagents may additionally comprise reagents capable of producing a second signal in the presence of an enzymatic activity in the reaction mixture. The second signal may be a chromogenic signal or a luminescent signal, such as a bioluminescent, chemiluminescent or fluorescent signal.

A chromogenic signal may be produced by the cleavage of a pro-chromogen molecule to release the chromogen. Suitable chromogens include indoxyl salts, such as 5-bromo-4-chloro-3-indoxyl-, 5-bromo-3-indoxyl-, 5-bromo-6-chloro-3-indoxyl-, 6-chloro-3-indoxyl-, N-methylindoxyl-, 2- or 4-nitrophenyl- and paranitroaniline. Suitable pro-chromogen molecules include X-Gal (bromo-chloro-indolyl-galactopyranoside) and o-nitrophenyl-beta-D-galactopyranoside (ONPG) which are commonly used for β-galactosidase detection, and variants thereof. Pro-chromogen molecules are normally added directly to the sample culture and the chromogenic signal is usually detectable after about 18 hours. The use of pro-chromogen molecules to detect bacteria is well-known in the art (see for example, Manafi et al. Microbiol. Mol. Biol. Rev., September 1991; 55: 335-348; Hansen et al J. Clin. Microbiol., December 1984; 20: 1177-1179; Haiyan Xu et al Appl. Envir. Microbiol., Dec. 1, 2007; 73: 7759-7762; James et al Appl. Envir. Microbiol. December 2000; 66: 5521-5523; Cassar et al J. Clin. Microbiol., July 2003; 41: 3229-3232).

A fluorometric signal may be produced by the cleavage of a pro-fluorophore molecule to release the fluorophore. Suitable fluorophores include methylumbelliferone and methylcoumarin. Suitable pro-fluorophores include 4-methylumbelliferyl-β-glucuronide, which is a β-glucuronidase substrate. The use of pro-fluorophore molecules to detect bacteria is well-known in the art well known in the art (see for example, Dahlén et al Appl Environ Microbiol. 1973 December; 26(6): 863-866; Vesley et al Appl. Envir. Microbiol; 58: 717-719; Karsten et al Appl. Envir. Microbiol. 1996; 62: 237-243; Clark et al Appl. Envir. Microbiol. 57: 1528-1534; Se-Wook Oh et al Appl. Envir. Microbiol. 2004; 70: 5692-5694)

A chemiluminescent signal may be produced by the cleavage of a pro-chemiluminescent molecule to release the chemiluminescent molecule. Suitable chemiluminescent molecules include dioxetanes, such as luminol. The use of pro-chemiluminescent molecules to detect bacteria is well-known in the art (see for example, Miller et al Appl. Envir. Microbiol; 35: 813-816; Stender et al Appl. Envir. Microbiol. 2001; 67: 142-147).

When the second signal is luminescent, it is preferably distinguishable from the first luminescent signal which is produced by the conversion of the pro-luciferin molecule into luciferin. For example, the first and second luminescent signals may have different wavelengths or may be produced under different conditions.

The second signal may be produced by the same enzymatic activity as the first luminescent signal. For example, detection of the second signal, which may be occur after detection of the first luminescent signal, may provide a confirmation of the presence of the target bacteria in the sample.

The second signal may be produced by a different enzymatic activity to the first luminescent signal.

The presence or amount of a first enzymatic activity in a sample may be determined by detecting the first luminescent signal (e.g. light produced at a first wavelength) and the presence or amount of a second enzymatic activity in the sample may be determined by detecting the second signal, which may be a chromogenic signal or a luminescent signal of a different wavelength to the first luminescent signal.

In some embodiments, the presence or amount of the first enzymatic activity in a sample may be determined by detecting a luminescent signal and the presence or amount of a second enzymatic activity in the sample may be determined by detecting a chromogenic signal. For example, a method of detecting coliforms may employ detection reagents comprising a luciferin- or coelenterazine-bound β-galactoside and a chromogenic β-galactoside marker (for example X-Gal). The first luminescent signal may be detected in 1 to 8 hours using a luminometer and the chromogenic signal may be detected after 24 hours, as a confirmation.

The use of a chromogenic marker may also be useful in the detection of pathogens. For example, a method of detecting E coli 0157 may comprise determining the presence or amount of β-galactosidase in a sample, for example after an incubation of 1 to 8 hours, by detecting a first luminescent signal and, subsequently, for example at 24 hours, determining the pathogenicity of the target bacteria in the sample by detecting a chromogenic signal, for example a signal produced by sorbitol fermentation or other 0157 chromogenic marker.

In some embodiments, the second signal may be a bioluminescent signal which has a different wavelength to the first luminescent signal or is otherwise distinguishable. For example, the detection reagents may comprise a pro-luciferin substrate for a first enzymatic activity and a pro-coelenterazine substrate for a second enzymatic activity. The presence of the first enzymatic activity may be determined by detecting a luciferin signal at about 490 nm and the presence of the second enzymatic activity may be determined by detecting a coelenterazine signal at about 590 nm. Luminescent signals at different wavelengths may be detected simultaneously to sequentially. For example, for the detection of E. coli, the detection reagents may comprise a luciferin-bound galactoside which produces a luminescent signal in the presence of β-galactosidase and a coelenterazine-bound glucuronate which produces a luminescent signal in the presence of β-glucuronidase. The detection of a luminescent signal at 490 nm and 590 nm is indicative of both β-galactosidase and β-glucuronidase activity in the sample culture and hence the presence of E. coli in the sample.

Dual Luciferase Assay Systems are known in the art (David S. McNabb, Robin Reed, and Robert A. Marciniak Eukaryot. Cell, September 2005; 4: 1539-1549; Tomoko Chiba-Mizutani et al, J. Clin. Microbiol. February 2007; 45: 477-487)

The first and second enzymatic activities may be produced by the same target bacteria and may provide confirmation of the presence of the target bacteria in the sample. For example, for the detection of E. coli, one of the luciferase signal and the second signal may be produced by β-glucuronidase activity and the other may be produced by β-galactosidase activity, as described above.

In other embodiments, the first and second enzymatic activities may be produced by different target bacteria. Detection of the first luminescent signal is thus indicative of the presence of first target bacteria in the sample and detection of the second luminescent signal is thus indicative of the presence of second target bacteria in the sample. The present methods may thus be used to detect two or more different target bacteria in a sample.

Different luminescent signals (e.g. light emitted at different wavelengths) may be produced by the different target bacteria. The presence or amount of a first target bacteria in a sample may be determined by detecting a first luminescent signal (e.g. light produced at a first wavelength) and the presence or amount of a second target bacteria in the sample may be determined by detecting a second luminescent signal (e.g. light produced at a second wavelength). The first luminescent signal is preferably a bioluminescent signal produced by luciferin generated from pro-luciferin to as described above. The second luminescent signal may be a bioluminescent, fluorescent or chemiluminescent signal.

Suitable bioluminescent, fluorescent or chemiluminescent substrates for the production of the second luminescent signal are well-known in the art.

For example, to detect two or more different target bacteria in the same sample, the detection reagents may comprise a pro-luciferin molecule which is specifically converted into luciferin by a first target organism, and a pro-coelenterazine molecule which is specifically converted into coelenterazine by a second target organism. The detection reagents or detection medium may further comprise a firefly luciferase which produces a first luminescent signal in the presence of luciferin, and a renilla luciferase which produces a second luminescent signal in the presence of coelenterazine. The first and second luminescent signals may be distinguished by their different wavelengths. Measurement of the first luminescent signal is therefore indicative of the presence or amount the first target bacteria in the sample and measurement of the second luminescent signal is therefore indicative of the presence or amount the second target bacteria in the sample.

Alternatively, a luminescent signal and a chromogenic signal may be produced by the different target bacteria. The presence or amount of first target bacteria in a sample may be determined by detecting the luminescent signal and the presence or amount of second target bacteria in the sample may be determined by detecting the chromogenic signal.

For example, detection reagents may comprise a luciferin-β-galactoside for the production of a luminescent signal if coliforms are present in the sample and a chromogenic factor based upon the reduction of a metabolic sugar for the production of a chromogenic signal, if E. coli. 0157 is present in the sample.

Suitable chromogenic substrates are well-known in the art and are described above.

In other embodiments, the detection reagents or detection medium may comprise a first pro-luciferin molecule which is specifically converted into luciferin by a first target organism, and a second pro-luciferin molecule which is specifically converted into luciferin by a second target organism. The differential growth of the first and second target bacteria may allow measurement of the luminescent signal at a first time point to be indicative of the presence or amount of the first target bacteria in the sample and measurement of the luminescent signal at a second time point to be indicative of the presence or amount of the second target bacteria in the sample.

Other aspects of the invention relate to methods of detecting target micro-organisms in a sample as described above except that the non-selective growth medium comprises detection reagents (without lysis reagents) before inoculation with the sample. For example, a method of detecting target micro-organisms in a sample may comprise;
a) providing a sample to be tested for target bacteria,
b) incubating the sample in a non-selective growth medium to produce a sample culture,
wherein the non-selective growth medium further comprises;
   (i) non-selective growth medium
   (ii) a pro-luciferin molecule which is specifically converted into luciferin by said target organism,
   (iii) luminescence reagents which produce a luminescent signal in the presence of luciferin; and
c) measuring the luminescent signal from the sample culture.

In some embodiments, the sample culture or portion thereof may be admixed with a buffer solution comprising a lysis reagent before the luminescent signal is measured. Suitable lysis reagents are described above.

The detection of a luminescent signal after incubation in the non-selective medium may be indicative of the presence of the target bacteria in the sample. For example, a luminescent signal which is above background levels (i.e. greater than controls without target bacteria), may be indicative that the target bacteria is present in the sample, whereas the absence of any luminescent signal above background levels may be indicative that the target bacteria is not present in the sample.

The amount of luminescent signal after incubation in the non-selective medium may be indicative of the number of target bacteria in the sample. For example, a high luminescent signal may be indicative of high numbers of target bacteria in the sample. For quantitation of numbers of target bacteria in the sample, the amount of luminescent signal may be measured from samples removed from the sample culture at two or more time points. The number of cells in the original sample may be extrapolated from the luminescent signals at the two or more time points as described below

Apart from the inclusion of detection reagents without lysis reagents in the non-selective medium, which removes the need for the addition of detection reagents after the incubation, methods according to these aspects of the invention are performed in the same way as described above.

Other aspects of the invention relate to devices suitable for performing methods of detecting target bacteria as described above.

In some embodiments, a single device may be used to perform the steps of the methods described above. For example, a detection device for target bacteria may comprise;
a sample chamber for housing a sampler,
a culture medium reservoir separated from the sample chamber by a first breakable barrier; and,
a detection reagent reservoir separated from the sample chamber or the culture medium reservoir by second breakable barrier,
such that the breakage of the first breakable barrier allows growth medium in the culture medium reservoir to enter the sample chamber, and breakage of the second breakable barrier allows detection reagents from the detection reagent reservoir to enter the sample chamber and/or culture medium reservoir.

In other embodiments, the steps of the methods described above may be used using a kit comprising two separate devices. For example, a detection kit for target bacteria may comprise;
a first device comprising;
   a sampler, such as a dipper or swab,
   a sample chamber for housing the sampler, and,
   a culture medium reservoir separated from the sample chamber by a first breakable barrier; such that the breakage of the first breakable barrier allows growth medium in the culture medium reservoir to enter the sample chamber; and,
a second device comprising
   a reaction chamber for accommodating a portion of sample culture from the first device; and,
   a detection reagent reservoir separated from the reaction chamber by a second breakable barrier,
such that the breakage of the second breakable barrier allows detection reagents contained in the detection reagent reservoir to enter the sample chamber.

In some embodiments, the second device may further comprise a buffer reservoir which is separated from the reaction chamber and/or the culture medium reservoir by a third breakable barrier, such that the breakage of the third breakable barrier allows buffer contained in the buffer chamber to enter the reaction chamber.

A reservoir preferably consists of an impermeable chamber or pouch which is contained within the housing of the device. The housing may be resiliently deformable to break the breakable barriers and allow egress of liquid from the reservoir.

Conveniently, the housing may be shaped to facilitate insertion into a luminometer to measure the luminescent signal. The housing may be transparent to facilitate the detection of luminescent signal and may optionally further comprise optically active regions, such as mirrors and lenses to amplify the luminescent signal for detection. For example, the housing may comprise a lens to focus and/or a mirror to reflect the luminescent signal emitted from the sample culture onto the detector in the luminometer.

In some embodiments, the culture medium reservoir may contain non-specific growth medium; the detection reagent reservoir may contain detection reagent; and/or the buffer reservoir may contain buffer. Growth medium, detection reagent and buffer are described above.

A breakable barrier is an impermeable barrier which prevents the passages of liquid medium or reagents and retains them within a reservoir or chamber. A breakable barrier may be disrupted by the user, for example by physical manipulation of the device, to allow the liquid medium or reagents to exit the reservoir. Suitable breakable barriers are well-known in the art and include foil or plastic membranes and snap-valves.

For example, a device may comprise a culture medium reservoir which is separated from the sample chamber by a membrane, such as a foil membrane. After sampling, the sampler is inserted into the device so as to pierce the membrane and become immersed in the growth medium in the culture medium reservoir. The device may then be incubated as described above.

Alternatively the culture medium reservoir may be in the form of a bulb which is connected to the sample chamber or detection chamber by a conduit which is closed by the first breakable barrier (termed a “snap-valve”). When the first breakable barrier is broken by the operator, for example by twisting or distorting the outer housing of the device, culture medium in the reservoir flows from the detection reagent reservoir through the conduit to the sample chamber, culture medium reservoir or detection chamber, where it contacts and immerses the sampler.

The detection reagent reservoir may be in the form of a bulb which is connected to the sample chamber or detection chamber by a conduit which is closed by the second breakable barrier (termed a “snap-valve”). When the second breakable barrier is broken by the operator, for example by twisting or distorting the outer casing of the device, the detection reagents flow from the detection reagent reservoir through the conduit to the sample chamber, culture medium reservoir or detection chamber, where they contact the sample culture. The detection reagent then kills the bacterial cells in the sample culture and initiates a bioluminescent reaction in the presence of enzymes from target bacteria which convert the pro-luciferin molecule into luciferin.

Suitable devices which may be adapted for use in the present methods are described in U.S. Pat. No. 5,266,266, U.S. Pat. No. 5,238,649, U.S. Pat. No. 5,078,968, U.S. Pat. No. 4,978,504, U.S. Pat. No. 6,248,294 and U.S. Pat. No. 5,869,003.

Another aspect of the invention provides the use of a bacterial detection device as described above in a method described above. In some embodiments, a method of detecting target bacteria may comprise;
providing a detection device for target bacteria as described above,
obtaining a sample using the sampler,
introducing the sample to the sample chamber,
breaking the first breakable barrier to introduce non-specific growth medium from the culture medium reservoir into the sample chamber,
incubating the sample in the non-specific growth medium in the sample chamber to produce the sample culture;
breaking the second breakable barrier to introduce detection reagents from the detection reagent reservoir into the sample chamber, and;
measuring the luminescent signal from the sample culture in the sample chamber.

In other embodiments, a method of detecting target bacteria may comprise;
providing a detection kit for target bacteria as described above,
obtaining a sample using the sampler of the first device,
introducing the sample to the sample chamber,
breaking the first breakable barrier to introduce non-specific growth medium from the culture medium reservoir into the sample chamber,
incubating the sample in the non-specific growth medium in the sample chamber to produce the sample culture;
transferring a portion of the sample culture to the reaction chamber of the second device,
breaking the second breakable barrier to introduce detection reagents from the detection reagent reservoir into the reaction chamber, and,
measuring the luminescent signal from the sample culture in the sample chamber.

A method may further comprise breaking the third breakable barrier to introduce buffer into the sample chamber or reaction chamber. This may be performed before or after exposure of the sample culture to the detection reagents.

Another aspect of the invention provides a method of producing a bacterial detection device comprising;
providing a bacterial detection device or kit as described above,
introducing detection reagent into the detection reagent reservoir; and
introducing non-specific growth medium into the culture medium reservoir.

In embodiments in which the device further comprises a buffer reservoir, the method may comprise introducing buffer into the buffer reservoir.

A suitable device may be adapted for use in a method of detecting target bacteria as described above.

Various further aspects and embodiments of the present invention will be apparent to those skilled in the art in view of the present disclosure. All documents mentioned in this specification are incorporated herein by reference in their entirety.

“and/or” where used herein is to be taken as specific disclosure of each of the two specified features or components with or without the other. For example “A and/or B″ is to be taken as specific disclosure of” each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described below.

FIG. 1 shows the increase in the detection of Enterobacter with increasing length of incubation using the bioluminescent beta-galactosidase substrate.

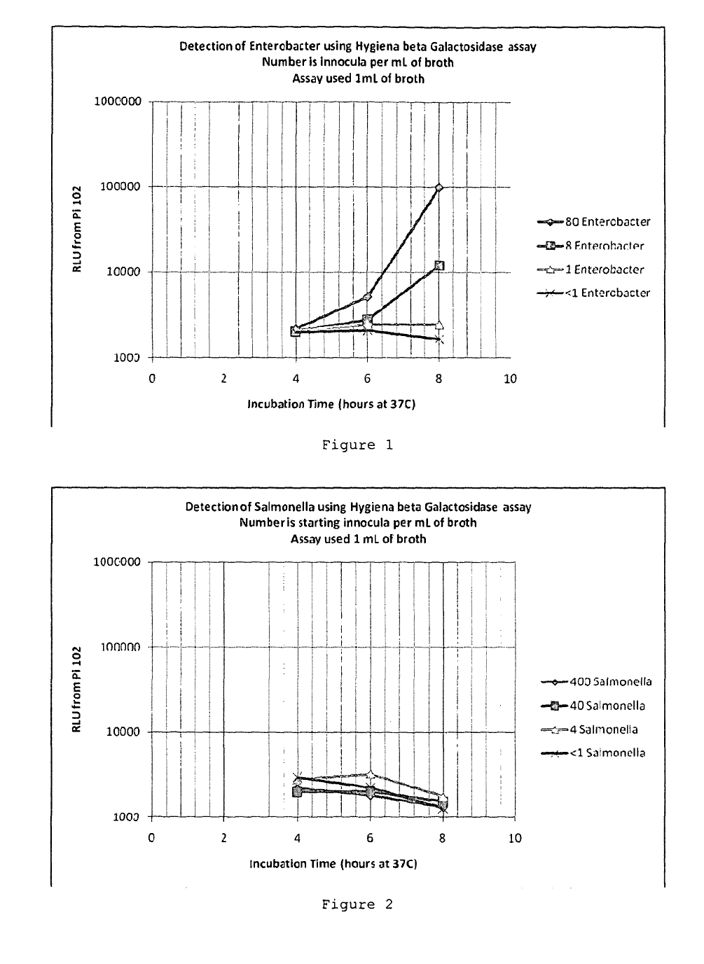

FIG. 2 shows that Salmonella is not detected using the bioluminescent beta-galactosidase substrate.

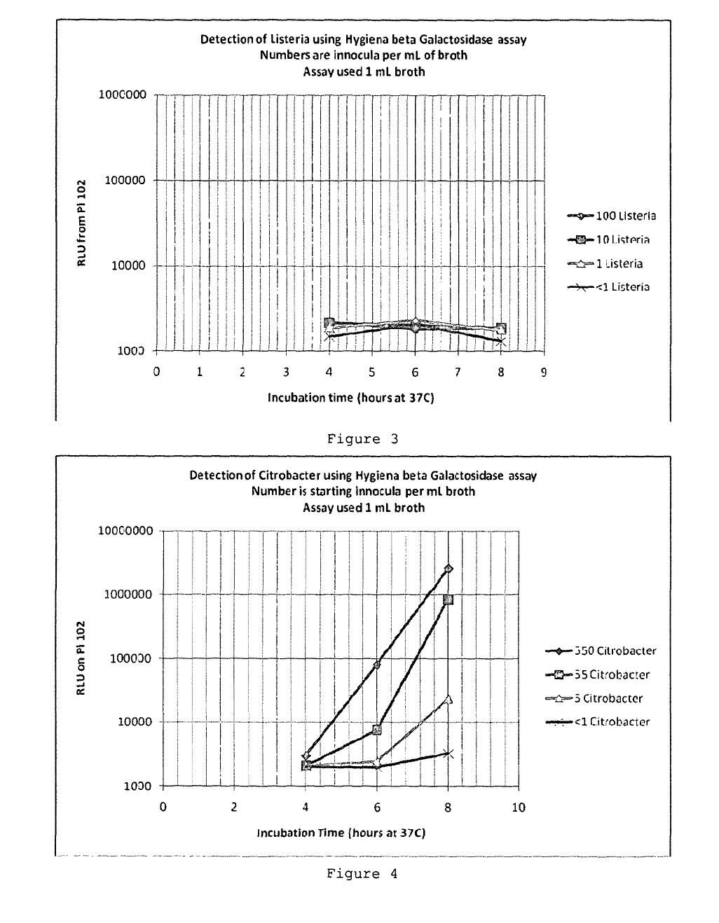

FIG. 3 shows that Listeria is not detected using the bioluminescent beta-galactosidase substrate.

FIG. 4 shows the increase in the detection of Citrobacter with increasing length of incubation using the bioluminescent beta-galactosidase substrate.

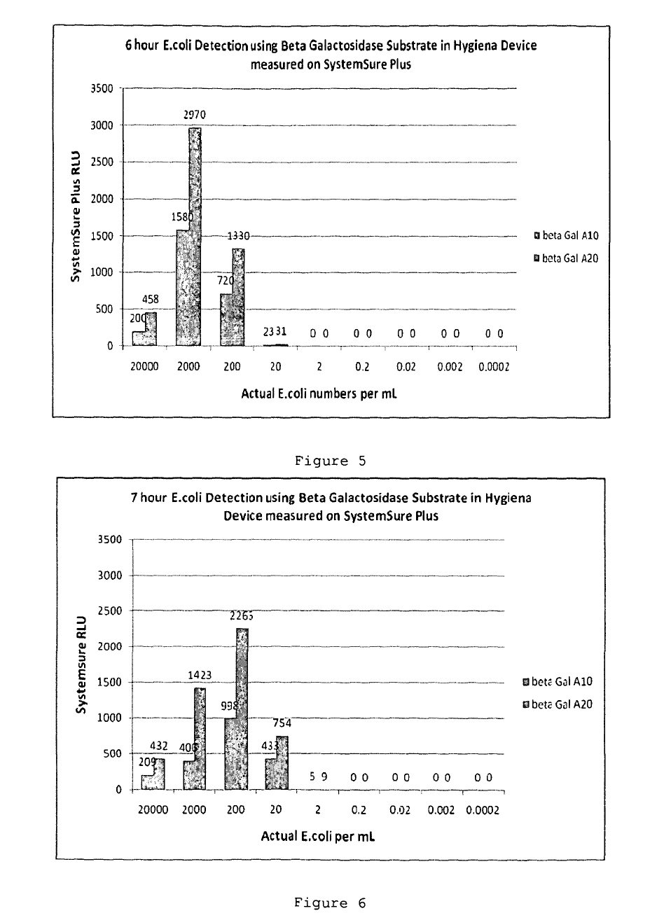

FIG. 5 shows the detection of dilutions of E. coli cells after 6 hours incubation using the bioluminescent beta-galactosidase substrate.

FIG. 6 shows the detection of dilutions of E. coli cells after 7 hours incubation using the bioluminescent beta-galactosidase substrate.

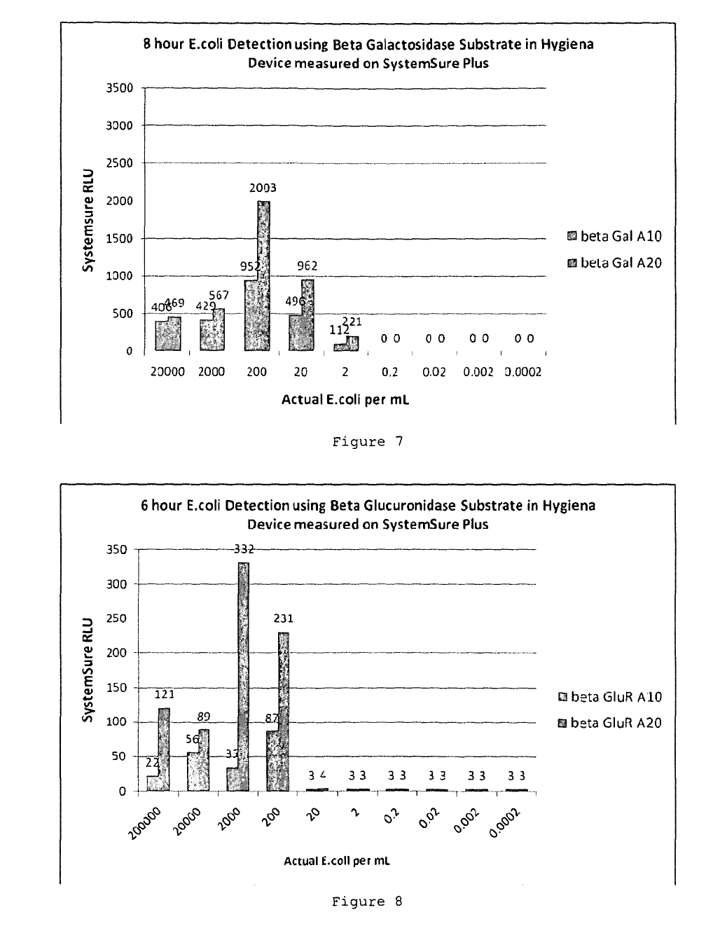

FIG. 7 shows the detection of dilutions of E. coli cells after 8 hours incubation using the bioluminescent beta-galactosidase substrate.

FIG. 8 shows the detection of dilutions of E. coli cells after 6 hours incubation using the bioluminescent beta-glucuronidase substrate.

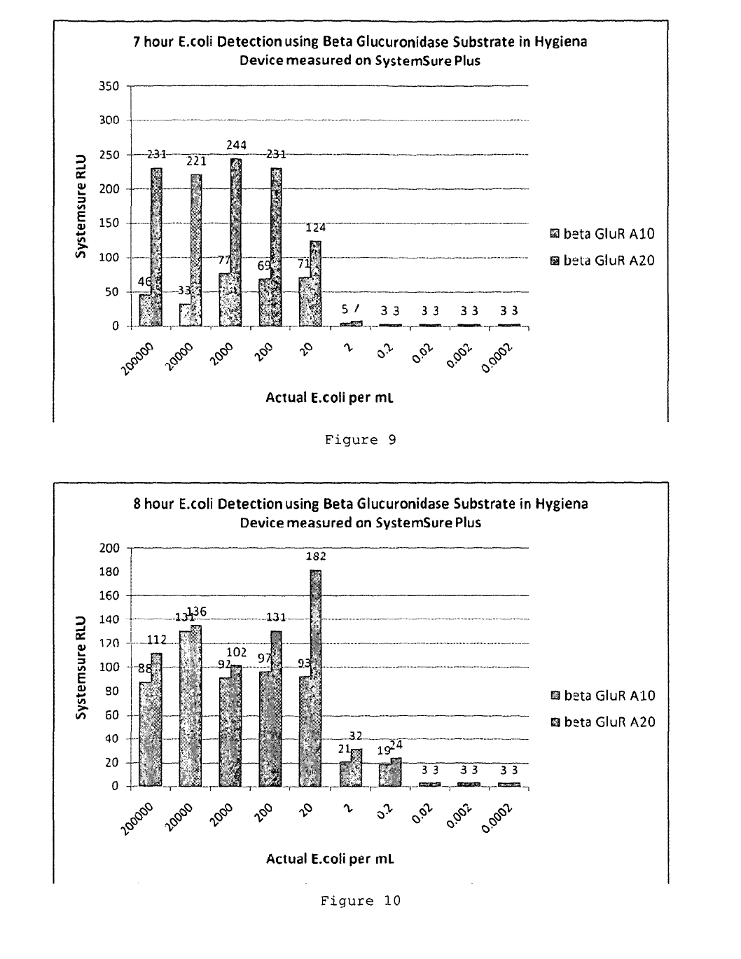

FIG. 9 shows the detection of dilutions of E. coli cells after 7 hours incubation using the bioluminescent beta-glucuronidase substrate.

FIG. 10 shows the detection of dilutions of E. coli cells after 8 hours incubation using the bioluminescent beta-glucuronidase substrate.

### EXPERIMENTS

### Bacteria

The bacteria in use in this study are shown in Table 1.

### Experiment 1

### Detection of Coliforms

### Method

Each coliform being assessed was grown overnight in 10 mL of sterile TSB (Tryptone Soya Broth) at 37 C static. The growth overnight was initiated from a purity plate also on TSA (Tryptone Soya Agar), after 18 hours incubation the culture was diluted to extinction into TSB broth supplemented with between 1 mM and 0.1 mM IPTG.

### Dilution Series

| Dilution | Expected bacteria | | |
|---|---|---|---|
| factor | per mL | High Range | Low Range |

| | | | |
|---|---|---|---|
| Neat | 1e9 | 5e9 | 1e8 |
| 10 | 1e8 | 5e8 | 1e7 |
| 100 | 1e7 | 5e7 | 1e6 |
| 1K | 1e6 | 5e6 | 1e5 |
| 10K | 1e5 | 5e5 | 1e4 |
| 100K | 1e4 | 5e4 | 1e3 |

| | | | | | | |
|---|---|---|---|---|---|---|
| 1M | 1e3 | (1000) | 5e3 | (5000) | 1e2 | (100) |
| 10M | 1e2 | (100) | 5e2 | (500) | 1e1 | (10) |
| 100M | 1e1 | (10) | 5e1 | (50) | 1e0 | (1) |
| 1KM | 1e0 | (1) | 5e0 | (5) | 1e−1 | (0.1) |
| 10KM | 1e−1 | (0.1) | 5e−1 | (0.5) | 1e−2 | (0.01) |

| | | | |
|---|---|---|---|
| Blank | 0 | 0 | 0 |

Typical broth volumes used for dilutions was 1-5 mL, for our purposes the lower volume of broth heats and maintains its temperature easier and quicker.

Each dilution series was then incubated for 2, 4, 6 and 8 hours at 37 C and the detection assay was then run at each time point on each dilution.

### Detection Reagents

UltraSnap™ reagent solution #164-consists of UltraSnap™ reagent as manufactured by MPC minus the addition of luciferin. To this is added 1 mM ATP and 10 ug/mL substrate mix (below).

Substrate beta-galactosidase K salt (PBI#3140A Lot #2-803-26) 5 mg was reconstituted to 1 mg/mL in pyrogen free water and aliquoted into 20 ul aliquots which were then frozen at −20 C until required. The optimum substrate concentration was deemed to be 0.01 mg/mL in the reagent above.

Buffer and Extraction Component—the buffer component was 100 mM Tris: BES pH 8.00 with the addition of 0.6% CHDG (Chlorohexidine digluconate). This component was used separately as an adjunct to correct the pH of the growing bacteria to a more favourable pH.

### Detection Assay

At each of the time points above 100 ul was removed from each of the dilutions. This was quickly added to 100 ul of buffer extraction component to terminate the bacterial growth and produce in effect an enzymatic cocktail.

To this was then added 100 ul of Ultrasnap™/beta Gal substrate mix, a stopwatch was then started and a reading of RLU taken in the Pi 102 PMT luminometer, a further 2 or 3 readings were taken of the same tubes at 10, 20 and 40 minutes (T0, T10, T20 and T40 min Assay Times)

The results are shown in FIGS. 1 to 4 and Tables 1 to 3. The initial results show detection at low levels of all Coliforms that were tested in these experiments; E. coli, Klebsiella, Enterobacter and Citrobacter were all detected at levels below 10 organisms. The confirmed detection time was between 6-8 hours for all these organisms. The 2 negative controls Salmonella and Listeria were not detected by the assay.

These results show that the present assay method can be used to quantify and identify coliform bacteria in as little as 6 hours at levels of detection approaching 10 organisms.

### Experiment 2

### Detection of Coliforms from Whole Chicken Wash

A highly contaminated natural meat product was used as source of wild type bacteria for use in comparison experiment of the methods described herein with known methods of coliform detection (3M Petrifilm™ and Nissui Compact Dry Media).

### Materials

A whole chicken (Tesco) was left at RT for 72 hours in original wrappings to increase natural flora of bacteria on surface and in peritoneal cavity. This was then washed with 250 mL of PFW, the whole chicken was placed in a sterile plastic bag with 250 mL of PFW, this was rinsed around for 10 minutes and then wash water was removed and chicken carcase then disposed of in rubbish.

### Coliform Collection Devices

Collection and incubation devices were manufactured with sufficient growth media to account for 4 assays from each device; the device will accurately deliver approximately 300 uL per test with the total broth volume being 1.2 mL. Additions to the broth volume of up to 500 uL of food diluents bring the total volume to 1.7 mL.

### Coliform Detection Devices

Detection devices were manufactured with sufficient detection reagents to perform 1 analysis for the enumeration of coliforms. These devices were set up to process 300 ul of broth delivered from the collection devices at specific time points in the incubation (2, 4, 6 and 8 hours post incubation at 37° C.).

Petrifilm™ used were the following Aerobic Count Plate, Coliform Count Plate, E. coli/Coliform Count Plate and Enterobacteriaceae Count Plate. Compact Dry used was Total Count, Coliform and E. coli/Coliform plates.

The wash water was stored at 4 C until required, but was used within 2 hours of washing chicken.

The chicken stock was then diluted in PFW in a decimal fold dilution series;

| Chicken | Coliform Collection Set - | |
|---|---|---|
| Dilution | up | Petrifilm/Compact Dry Set-up |
| Neat | 500 uL added to 2x Coliform | NA |
| | Growth Device | |
| Dilution - | 500 uL added to 2x Coliform | NA |
| 1 | Growth Device | |
| Dilution - | 500 uL added to 2x Coliform | NA |
| 2 | Growth Device | |
| Dilution - | 500 uL added to 2x Coliform | 1 mL added to Petrifilm and |
| 3 | Growth Device | Compact Dry plates |
| Dilution - | 500 uL added to 2x Coliform | 1 mL added to Petrifilm and |
| 4 | Growth Device | Compact Dry plates |
| Dilution - | 500 uL added to 2x Coliform | 1 mL added to Petrifilm and |
| 5 | Growth Device | Compact Dry plates |
| Dilution - | 500 uL added to 2x Coliform | 1 mL added to Petrifilm and |
| 6 | Growth Device | Compact Dry plates |
| Dilution - | 500 uL added to 2x Coliform | 1 mL added to Petrifilm and |
| 7 | Growth Device | Compact Dry plates |
| Dilution - | 500 uL added to 2x Coliform | NA |
| 8 | Growth Device | |
| Dilution - | 500 uL added to 2x Coliform | NA |
| 9 | Growth Device | |
| Dilution - | 500 uL added to 2x Coliform | NA |
| 10 | Growth Device | |

Coliform Collection Devices were set-up to have enough devices to run duplicate assays at 2, 4, 6 and 8 hours after incubation at 37 C. All Petrifilm™ and Compact Dry plates were also incubated at 37 C. At each time point 300 ul was squeezed from the Coliform Collection Devices into a Coliform Detection Device the Coliform Detection Device was then incubated at 37 C in a heating block and assayed for light production at 10, 20 and 60 minutes post-activation of the device.

Each device was concurrently assayed in 2 luminometers for light output, the SystemSure Plus™ and the Pi 102. Both luminometers have different sensitivities with the Pi 102 being approximately 600 to 1000 more sensitive than the Systemsure plus.

The results are displayed in Tables 5, 6 and 7 below. Each table has RLU values for SystemSure Plus™ first then Pi102 subsequently. The sets of results show 10 minute to 60 minute assay times, with each table describing each 2, 4, 6 and 8 hour incubation times at 37 C.

Tables 5, 6 and 7 show the results with 10, 20 and 60 minute assay times, respectively, with 2, 4, 6 and 8 hour incubation times for coliform collection devices incubating dilutions of Whole Chicken Wash. The inoculum size was estimated from Petrifilm™ counts from EC and CC plates after 24 hours incubation. The numbers in bold are limits of detection.

For a 10 minutes assay time, the SystemSURE™ luminometer detected less than 10 organisms per ml after 6 hour incubation. This was a confirmed positive, if the incubation time was increased to 8 hours at 37 C. At 8 hours incubation, the detection limit also dropped to a confirmed single organism or less.

For a 10 minutes assay time, the Pi102 luminometer detected a confirmed positive of 1 or less organism per ml. This was confirmed by increasing the incubation time to 8 hours, but although this increased the signal RLU, it did not increase the detection level.

As the assay time was increased, the detection of low numbers of organisms became easier, as the signal RLU rose well above the blank values. The signal to noise ratio was not affected to the detriment of the test and positives on both the SystemSURE™ and the Pi102 were distinguished easily.

The counts from the Petrifilm™ and the Compact Dry were very similar for both the Coliform only and the E. coli/Coliform agar types. There was a slight differentiation in the Total Count media with the Petrifilm™ seemingly growing more bacteria than the Total Count Compact Dry, although this may have been the result of nutrient differences in these media or the ability of growing organisms to utilise the redox dyes used as biomarkers.

### Experiment 3

### Detection of E. coli ATCC 25962 Over a Time Course of 6, 7 and 8 Hours

The detection of E. coli as both a Coliform using beta galactosidase and as E. coli using beta glucuronidase was tested over an 8 hour incubation period.

### Method

TSB (OXOID) was made up in sterile water and sterile filtered. This method gave lower blanks in the test and in this final experiment the blank for the beta galactosidase reaction was lowered to 0 (Zero) on the SystemSure and 3 (three) for beta glucuronidase on the SystemSure.

The detection reagent was Ultrasnap™ formulation without Luciferin; this is supplemented with NRM at working strength by dissolving benethonium chloride into the UltraSnap™ directly.

Beta galactosidase and beta-glucuronidase substrate was added at a concentration of 0.001 ug/mL from a stock of 1 mg/mL in water. 2.5 mM ATP is added from a stock solution of 100 mM ATP also in water. The stock solutions are kept frozen until required.

In order to maintain the stability of the Ultrasnap™ in a form as close to the original formulation, the reaction was rebuffered from a separate chamber. The bulb chamber held the bulk of the Ultrasnap™+ATP+substrate+NRM reagent (volume 500 uL), while the small secondary chamber held the rebuffering solution (100-150 uL) which rebuffered the reaction to pH 8.0 around a concentration of 30 mM Tris HCl or Tris Tricine.

500 ul of broth consisting of sterile-filtered TSB with 0.5 mM IPTG was held in a foiled chamber at the bottom of the device. A known inducer for beta glucuronidase (methyl beta glucuronide) was tested and found not to be beneficial in the assay. The swab was wetted with a neutral wetting agent, such as MRD.

### SystemSure Experiments

E. coli was grown static in TSB overnight at 37° C. and then directly diluted into 12×1 ml volume of TSB+0.5 mM IPTG in a serial decimal dilution series. The overnight count from the static E. coli was counted via the Miles and Misra method to be 2e8 per mL. The dilution series was then as follows;

| | | |
|---|---|---|
| Dilution | E. coli | |
| factor | per mL | Assayed |

| | | | | |
|---|---|---|---|---|
| 1 (Neat) | 2e8 | 6 hours | 7 hours | 8 hours |
| 10-1 | 2e7 | 6 hours | 7 hours | 8 hours |
| 10-2 | 2e6 | 6 hours | 7 hours | 8 hours |
| 10-3 | 2e5 | 6 hours | 7 hours | 8 hours |
| 10-4 | 20000 | 6 hours | 7 hours | 8 hours |
| 10-5 | 2000 | 6 hours | 7 hours | 8 hours |
| 10-6 | 200 | 6 hours | 7 hours | 8 hours |
| 10-7 | 20 | 6 hours | 7 hours | 8 hours |
| 10-8 | 2 | 6 hours | 7 hours | 8 hours |
| 10-9 | 0.2 | 6 hours | 7 hours | 8 hours |
| 10-10 | 0 | 6 hours | 7 hours | 8 hours |
| 10-11 | 0 | 6 hours | 7 hours | 8 hours |
| 10-12 | 0 | 6 hours | 7 hours | 8 hours |

The assay was run as a ratio of 1:1 with broth and detection reagent, either as 500 ul:500 ul or in lower volume of the same ratio.

The higher concentrations of E. coli at 2e5 and above demonstrated severe inhibition of the assay due to the turbid growth after 6, 7 and 8 hours. However, this inhibition never fell below a certain level and was never low enough to be detected as a false negative.

The assay time shown as beta Gal A10 and beta Gal A20 was the time between activation of the device after the incubation period and the time it is read in the luminometer. This time needs to be at least 10 minutes.

The actual RLU values shown in the results demonstrated the difference in expression levels of beta galactosidase and beta glucuronidase. The difference in expression rates was approximately 10 to 20 fold more enzyme, although this is a rough figure form the data and the exact expression difference is around 100× fold.

### Results

The results were expressed as plots of mean RLU versus actual E. coli numbers, each graph taken at different time points (FIGS. 6 to 11), the first at 6 hours, the second at 7 hours and the third at 8 hours. The assay times run were 10 and 20 minutes both are shown on the graphs.

FIG. 6 shows a 6 hour graph for beta-galactosidase with the detection of 20 E. coli just emerging from the baseline, around 23 RLU at 10 minute assay time, which increased to 31 RLU at 20 minute assay time. The low RLUs at 20,000 E. coli level demonstrated the depression in light output from the assay. At the higher levels of E. coli, the RLU for 10 minute assays were also found to be depressed, although the 2e7 E. coli level still exhibited 120 RLUs, which was detectable.

FIG. 7 shows a 7 hours graph for beta galactosidase, with the signal for the detection of 2 E. coli just emerging from the baseline, around 5 RLU at 10 minute assay time, which increased to 9 RLU at 20 minute assay time.

FIG. 8 shows an 8 hours graph for beta galactosidase with the signal for the detection of 2 E. coli now well above the baseline at around 112 RLU at 10 minute assay time, which increased to 221 RLU at 20 minute assay time. This extreme level of detection becomes a statistical certainty at between 7 and 8 hours, the detection of 1 or 2 E. coli bacteria can then become a differential test easily done in a shift using simple devices without having to recourse to complex sub-culturing methods

FIG. 9 shows a 6 hours graph for beta glucuronidase which shows the detection of 20 E. coli just emerging from the baseline, around 4 RLU at 20 minute assay time, although the blank values are at 3, so the detection of 20 E. coli was unreliable using beta glucuronidase at 6 hours. However, 200 E. coli were easily detected at 6 hours, although the RLU levels were 10 times lower for this enzyme than would be for a similar galactosidase level.

FIG. 9 shows a 7 hours graph for beta glucuronidase which shows the detection of 2 E. coli just emerging from the baseline, around 5 RLU at 10 minute assay time with and increase to 9 RLU at 20 minute assay time. 20 E. coli were picked up in 7 hours using beta glucuronidase, although the 2 E. coli signal was not significantly higher than the blanks.

FIG. 9 shows an 8 hours graph for beta glucuronidase which shows the detection of 2 E. coli well above the baseline around 21 RLU at 10 minute assay time, which increased to 32 RLU at 20 minute assay time. Since one of the replicates at the next dilution series 0.2 per mL started to grow, this must also have contained 1 E. coli cell. Although the extreme level of detection becomes a statistical certainty at between 7 and 8 hours, the use of glucuronidase resulted in a slightly later detection level and lower RLUs than the corresponding galactosidase assay.

The above results show that both coliforms and E. coli may be detected rapidly and sensitively using bioluminogenic substrates for beta galactosidase and beta glucuronidase.

The detection level of coliforms using beta-galactosidase was found to be; 20-200 bacteria at 6 hours, 2-20 bacteria at 7 hours and <2 bacteria confirmed at 8 hours.

The detection level of E. coli using beta Glucuronidase was found to be; >200 bacteria at 6 hours, 20-200 bacteria at 7 hours and <2 bacteria confirmed at 8 hours.

The RLU values for a coliform in exponential phase were found to be around 2000 RLU on the SystemSure, whereas the RLU values for E. coli in exponential phase were found to be around 300 RLU on the SystemSure

The blank values for Coliforms were all 0 RLU and the blank values for E. coli were all 3 RLU.

### Experiment 4

Table 9 shows an algorithm for semi-quantitative enumeration derived from multiple passes of type bacteria from a culture of Enterobacteriacae.

Table 10 shows the application of the derived algorithm to real data from Chicken and Mince to estimate the accuracy and range of the detection of bacteria when compared to a standard method (3M Petrifilm).

### Experiment 5

Approximately 1000 cells of different types of bacteria were grown in 2 mL of appropriate broth—TSB or BHI for 8 hours at 37 C.

100 μL of the sample broth was then removed and 100 μL lysis reagent (NRM™ reagent; reference—solution 0063; Hygiena Int, CA) added to it.

400 uL of detection reagent was then added to the lysed cells and the bioluminescent output was measured in a SystemSure™ Plus luminometer (Hygiena Int, CA) over a 10 minute assay time.

The detection reagents used for each test are shown in Table 11 and the bioluminescence (in RLUs) recorded from each test on each type of bacteria is shown in Table 12. It is evident from this data that the bioluminescent signals produced by these tests can be used to differentiate and type bacteria.

The ability of each test to detect different bacteria is summarised in Table 13. It is evident that each type of bacteria has a specific bioluminogenic biochemical fingerprint which can be detected in less than 8 hours.

TABLE 1

| Type Bacterial Strain | Type Number or Code | Characteristics |
|---|---|---|
| Bacillus subtilis | ATCC 6633 | G(+) control |
| Campylobacter jejuni | ATCC 33291 | G(+) control |
| Candida albicans | ATCC 10231 | Yeast control |
| Citrobacter freundii | ATCC 8090 | Coliform |
| Citrobacter freundii | NCTC 8168 | Coliform |
| Enterobacter | ATCC 13048 | Coliform |
| aerogenes | | |
| Enterobacter cloacae | NCTC 8168 | Coliform |
| Enterococcus faecalis | NCTC 8213 | G(+) cocci |
| Enterococcus faecalis | ATCC 33186 | G(+) cocci |
| Escherichia coli | NCTC 9001 | Faecal Coliform/ |
| | | Coliform |
| Escherichia coli | ATCC 25922 | Faecal Coliform/ |
| | | Coliform |
| Klebsiella aerogenes | C2126 | Coliform |
| Listeria fermentum | ATCC 9338 | G(+) control |
| Listeria ivanovii | ATCC 19119 | G(+) control |
| Listeria welshimeri | ATCC 35897 | G(+) control |
| Pseudomonas | ATCC 9027 | G(−) control |
| aeruginosa | | |
| Salmonella | ATCC 10708 | G(−) control |
| choleraesuis | | |
| Staphylococcus aureus | ATCC 6538 | G(+) cocci |
| Streptococcus | ATCC 19615 | G(+) cocci |
| pyogenes | | |
| Wild Types | | |
| K#1 | Gal(−)GluR(−) GluC(−) | Non Enteric Bacteria |
| K#2 | Gal(+)GluR(−) GluC(−) | Coliform |
| K#3 | Gal(+) GluR(−)GluC(+) | Klebsiella |
| K#4 | Gal(+) GluR(−) GluC(−) | Coliform |
| K#5 | Gal(+) GluR(−) GluC(−) | Coliform |
| LT#1 | Gal(+) GluR(+) GluC(−) | E. coli |
| LT#2 | Gal(+) GluR(−) GluC(−) | Coliform |
| LT#3 | Gal(+) GluR(−) GluC(−) | Coliform |
| LT#4 | Gal(−) GluR(−) GluC(−) | Non Enteric Bacteria |
| LT#5 | Gal(−) GluR(−) GluC(−) | Non Enteric Bacteria |
| LR#1 | Gal(−) GluR(−) GluC(−) | Non Enteric Bacteria |
| LR#2 | Gal(+) GluR(−) GluC(−) | Coliform |
| LR#3 | Gal(+) GluR(−) GluC(−) | Coliform |
| LR#4 | Gal(−) GluR(−) GluC(−) | Non Enteric Bacteria |
| LR#5 | Gal(−) GluR(−) GluC(−) | Non Enteric Bacteria |

TABLE 2
4 hour incubation of Coliforms and E. coli
at 37 C. Assay time 0, 30 and 60 minutes

| | Starting | Assay time | Assay time | Assay time |
|---|---|---|---|---|
| | Innocula | 0 | 30 mins | 60 mins |
| E. coli | 900 | 60541 | 7248 | 4900 |
| | 90 | 53409 | 2200 | 1090 |
| | 9 | 54232 | 2317 | 1123 |
| | 1 | 55212 | 2127 | 1098 |
| Klebsiella | 1200 | 57481 | 18902 | 11684 |
| | 120 | 55439 | 3234 | 1678 |
| | 12 | 48790 | 1879 | 897 |
| | 1 | 54390 | 2236 | 1023 |
| Enterobacter | 80 | 56588 | 2160 | 951 |
| | 8 | 55417 | 2890 | 1214 |
| | 1 | 49800 | 2090 | 1134 |
| | <1 | 51290 | 1980 | 1020 |
| Salmonella | 400 | 56528 | 2224 | 897 |
| | 40 | 52180 | 1985 | 1211 |
| | 4 | 49822 | 2800 | 1235 |
| | <1 | 46721 | 2982 | 1384 |
| Listeria | 100 | 55920 | 2200 | 900 |
| | 10 | 53290 | 2180 | 1134 |
| | 1 | 55239 | 1908 | 1221 |
| | <1 | 55329 | 1498 | 902 |
| Citrobacter | 550 | 55557 | 3018 | 1534 |
| | 55 | 48712 | 2109 | 1134 |
| | 5 | 53800 | 2090 | 1090 |
| | <1 | 49845 | 2042 | 1209 |

TABLE 3
6 hour incubation of Coliforms and E. coli
at 37 C. Assay time 0, 30 and 60 minutes

| | Starting | Assay time | Assay time | Assay time |
|---|---|---|---|---|
| | Innocula | 0 | 30 mins | 60 mins |
| E. coli | 900 | 49647 | 278790 | 279324 |
| | 90 | 49842 | 32735 | 31023 |
| | 9 | 50177 | 4609 | 3250 |
| | 1 | 50812 | 2090 | 1021 |
| Klebsiella | 1200 | 34342 | 3127903 | 2816386 |
| | 120 | 43622 | 229494 | 221406 |
| | 12 | 47042 | 9908 | 6688 |
| | 1 | 50902 | 2568 | 1521 |
| Enterobacter | 80 | 44556 | 5157 | 4182 |
| | 8 | 47289 | 2039 | 1145 |
| | 1 | 48186 | 2461 | 867 |
| | <1 | 49865 | 2098 | 1023 |
| Salmonella | 400 | 45660 | 1832 | 748 |
| | 40 | 43966 | 2025 | 900 |
| | 4 | 43479 | 3233 | 892 |
| | <1 | 45907 | 2287 | 993 |
| Listeria | 100 | 47176 | 1845 | 773 |
| | 10 | 46906 | 2020 | 885 |
| | 1 | 46637 | 2342 | 898 |
| | <1 | 46790 | 2090 | 998 |
| Citrobacter | 550 | 44362 | 80788 | 79940 |
| | 55 | 44025 | 7706 | 6346 |
| | 5 | 43082 | 2468 | 1235 |
| | <1 | 44509 | 2005 | 1090 |

TABLE 4
8 hour incubation of Coliforms and E. coli
at 37 C. Assay time 0, 30 and 60 minutes

| | Starting | Assay time | Assay time | Assay time |
|---|---|---|---|---|
| | Innocula | 0 | 30 mins | 60 mins |
| E. coli | 900 | 340662 | 2482912 | 2209012 |
| | 90 | 1678767 | 3116542 | 2900565 |
| | 9 | 76387 | 112540 | 110900 |
| | 1 | 29187 | 26300 | 26000 |
| Klebsiella | 1200 | 3954639 | 5201026 | 4990900 |
| | 120 | 2856767 | 4351622 | 4098122 |
| | 12 | 735731 | 1132432 | 1120090 |
| | 1 | 40291 | 37436 | 36790 |
| Enterobacter | 80 | 52486 | 100057 | 99023 |
| | 8 | 19270 | 12121 | 12900 |
| | 1 | 13319 | 2460 | 1200 |
| | <1 | 10176 | 1651 | 1000 |
| Salmonella | 400 | 21390 | 1331 | 1000 |
| | 40 | 14804 | 1542 | 905 |
| | 4 | 11933 | 1762 | 1023 |
| | <1 | 14032 | 1291 | 980 |
| Listeria | 100 | 16116 | 1910 | 1200 |
| | 10 | 12225 | 1897 | 1312 |
| | 1 | 11490 | 1787 | 976 |
| | <1 | 10293 | 1329 | 998 |
| Citrobacter | 550 | 1066561 | 2678025 | 2645902 |
| | 55 | 475339 | 862267 | 854900 |
| | 5 | 26178 | 23938 | 22189 |
| | <1 | 13711 | 3277 | 2567 |

TABLE 5

| | | 10 minute assay time | | |
|---|---|---|---|---|
| | | Incubation time at 37 C. | | |
| Inoculum size | 2 hours | 4 hours | 6 hours | 8 hours |

Systemsure plus

| | | | | |
|---|---|---|---|---|
| 1.00E+07 | 539 | 739 | 983 | 1211 |
| 1000000 | 218 | 375 | 1069 | 1577 |
| 100000 | 1 | 209 | 418 | 1635 |
| 10000 | 2 | 171 | 434 | 1274 |
| 1000 | 0 | 2 | 503 | 821 |
| 100 | 3 | 0 | 501 | 1183 |
| 10 | 4 | 1 | 10 | 604 |
| 1 | 3 | 2 | 0 | 10 |
| 0.1 | 3 | 1 | 0 | 0 |
| 0.01 | 4 | 1 | 0 | 0 |
| 0.001 | 4 | 1 | 0 | 0 |

Pi 102

| | | | | |
|---|---|---|---|---|
| 1.00E+07 | 311253 | 731610 | 686768 | 850122 |
| 1000000 | 125715 | 371250 | 747231 | 1106703 |
| 100000 | 2303 | 206415 | 291833 | 1147770 |
| 10000 | 867 | 169290 | 303017 | 893997 |
| 1000 | 66 | 769 | 351597 | 575991 |
| 100 | 55 | 177 | 349850 | 830115 |
| 10 | 57 | 67 | 7591 | 424008 |
| 1 | 56 | 45 | 166 | 6669 |
| 0.1 | 55 | 55 | 75 | 506 |
| 0.01 | 76 | 64 | 72 | 58 |
| 0.001 | 66 | 34 | 66 | 59 |

TABLE 6

| | | 10 minute assay time | | |
|---|---|---|---|---|
| | | Incubation time at 37 C. | | |
| Inoculum size | 2 | 4 | 6 | 8 |

Systemsure plus

| | | | | |
|---|---|---|---|---|
| 1.00E+07 | 994 | 1264 | 1580 | 1832 |
| 1000000 | 463 | 683 | 1854 | 2590 |
| 100000 | 77 | 441 | 809 | 2711 |
| 10000 | 7 | 368 | 799 | 2164 |
| 1000 | 7 | 7 | 939 | 1437 |
| 100 | 7 | 7 | 941 | 1931 |
| 10 | 8 | 7 | 67 | 1115 |
| 1 | 7 | 5 | 1 | 32 |
| 0.1 | 7 | 5 | 0 | 2 |
| 0.01 | 8 | 5 | 1 | 0 |
| 0.001 | 7 | 5 | 1 | 0 |

Pi 102

| | | | | |
|---|---|---|---|---|
| 1.00E+07 | 797188 | 1102208 | 1189364 | 1084461 |
| 1000000 | 348176 | 595576 | 1414602 | 1519509 |
| 100000 | 58829 | 344471 | 560291 | 1604203 |
| 10000 | 1295 | 295136 | 505767 | 1353837 |
| 1000 | 77 | 4532 | 660117 | 840373 |
| 100 | 65 | 462 | 595653 | 1180511 |
| 10 | 72 | 45 | 47101 | 725273 |
| 1 | 66 | 77 | 367 | 24090 |
| 0.1 | 63 | 66 | 133 | 3491 |
| 0.01 | 71 | 64 | 66 | 70 |
| 0.001 | 57 | 76 | 55 | 72 |

TABLE 7

| | | 60 minute assay time | | |
|---|---|---|---|---|
| | | Incubation time at 37 C. | | |
| Inoculum size | 2 | 4 | 6 | 8 |

Systemsure plus

| | | | | |
|---|---|---|---|---|
| 1.00E+07 | 1654 | 1798 | 1406 | 1003 |
| 1000000 | 909 | 1451 | 2961 | 2105 |
| 100000 | 147 | 979 | 1557 | 3353 |
| 10000 | 2 | 816 | 1460 | 3496 |
| 1000 | 0 | 14 | 1756 | 2765 |
| 100 | 0 | 2 | 1658 | 3474 |
| 10 | 2 | 0 | 181 | 2169 |
| 1 | 2 | 0 | 0 | 111 |
| 0.1 | 3 | 0 | 0 | 31 |
| 0.01 | 3 | 0 | 0 | 0 |
| 0.001 | 4 | 1 | 1 | 0 |

Pi 102

| | | | | |
|---|---|---|---|---|
| 1.00E+07 | 1655654 | 1412835 | 1408090 | 10460 |
| 1000000 | 909909 | 1140093 | 1800322 | 731834 |
| 100000 | 147147 | 769101 | 1048113 | 1913095 |
| 10000 | 2002 | 641376 | 1048650 | 1987430 |
| 1000 | 69 | 11004 | 1193653 | 1680202 |
| 100 | 55 | 1179 | 1057696 | 1912805 |
| 10 | 57 | 77 | 136686 | 1291487 |
| 1 | 58 | 77 | 624 | 77991 |
| 0.1 | 66 | 68 | 179 | 21302 |
| 0.01 | 76 | 81 | 53 | 70 |
| 0.001 | 77 | 56 | 63 | 81 |

TABLE 8

| | % Chance of Detection at p < 0.05 | | | | | |
|---|---|---|---|---|---|---|
| Time Period | Initial inoculum size bacteria per swab | | | | | |
| (hours) of | >1 | >10 | >100 | >1000 | >10000 | >100,000 |
| Incubation | Coli- | Coli- | Coli- | Coli- | Coli- | Coli- |
| at 37 C. | forms | forms | forms | forms | forms | forms |
| 2 | 0 | 0 | 0 | 50 | 95 | 99 |
| 4 | 0 | 0 | 90 | 95 | 99 | 99 |
| 6 | 25 | 50 | 95 | 99 | 99 | 99 |
| 7 | 90 | 95 | 99 | 99 | 99 | 99 |
| 8 | 95 | 99 | 99 | 99 | 99 | 99 |

TABLE 9
Pi 102

| 2-4 hours | | Linear Range |
|---|---|---|
| 10 minute assay | y = 0.0551x to 1.2883 | (10-100000) |
| 20 minute assay | y = 0.0443x to 1.3869 | (10-100000) |
| 60 minute assay | y = 0.2597x to 1.3114 | (10-100000) |

y = RLU
x = Initial Count

TABLE 10

| | Algorithm | Petrifilm | Actual | Actual |
|---|---|---|---|---|
| Pi 102 | estimate | Estimate | 2 hour RLU | 4 hour RLU |
| Chicken (2-4) 20 | | | | |
| minute assay | | | | |
| 293841 | 41461 | 10000 | 1295 | 295136 |
| 4455 | 2023 | 1000 | 77 | 4532 |
| 397 | 354 | 100 | 65 | 462 |
| Mince (2-4) 20 | | | | |
| minute assay | | | | |
| 884736 | 91792 | 40000 | 10647 | 895383 |
| 75143 | 15511 | 4000 | 181 | 75323 |
| 507 | 422 | 400 | 79 | 585 |
| 20 | 41 | 40 | 61 | 81 |

TABLE 11
Reagent for Detection of Bacterial Genera

| E. coli Detection Reagent | | |
|---|---|---|
| UltraSnap minus Luciferin (proprietary Hygiena reagent) | 10 | mL |
| Adenosine Triphosphate | 1 | mM |
| Luciferin-Glucuronide | 1 | ug/mL |
| Coliform Detection Reagent | | |
| UltraSnap minus Luciferin (proprietary Hygiena reagent) | 10 | mL |
| Adenosine Triphosphate | 1 | mM |
| Luciferin-Galactoside | 1 | ug/mL |
| Listeria monocytogenes Detection Reagent | | |
| UltraSnap minus Luciferin (proprietary Hygiena reagent) | 10 | mL |
| Adenosine Triphosphate | 1 | mM |
| Luciferin-o-phosphatidylinositol | 1 | ug/mL |
| Listeria species/Enterococcus/Yersinia Detection Reagent | | |
| UltraSnap minus Luciferin (proprietary Hygiena reagent) | 10 | mL |
| Adenosine Triphosphate | 1 | mM |
| Luciferin-Glucoside | 1 | ug/mL |
| Protease Detection Reagent | | |
| UltraSnap minus Luciferin (proprietary Hygiena reagent) | 10 | mL |
| Adenosine Triphosphate | 1 | mM |
| Luciferin-Alanine-Alanine-Phenylalanine | 1 | ug/mL |
| Phosphatase Detection Reagent | | |
| UltraSnap minus Luciferin (proprietary Hygiena reagent) | 10 | mL |
| Adenosine Triphosphate | 1 | mM |
| Luciferin-o-phosphate | 1 | ug/mL |

TABLE 12

| | | | | Listeria | | | |
|---|---|---|---|---|---|---|---|
| | | E. coli | Coliform | mono | Enterococcus | Protease | Phosphatase |
| 1 | MRSA 2 | 0 | 0 | 0 | 0 | 0 | 2922 |
| 2 | MRSA 4 | 0 | 0 | 0 | 0 | 0 | 2163 |
| 3 | MRSA 5 | 2 | 0 | 0 | 3 | 0 | 4134 |
| 4 | L. ivanovii | 0 | 3 | 104 | 49 | 0 | 31 |
| 6 | MRSA 1 | 1 | 0 | 0 | 0 | 0 | 2674 |
| 7 | E coli 9001 | 1818 | 1085 | 66 | 158 | 8 | 42 |
| 8 | E coli O157 | 1 | 553 | 86 | 20 | 12 | 109 |
| 9 | Citrobacter | 1 | 435 | 99 | 37 | 2 | 44 |
| 10 | E coli 8739 | 4324 | 491 | 74 | 270 | 4 | 76 |
| 11 | Finger Dry | 2 | 4 | 0 | 160 | 2 | 1236 |
| 12 | Finger staph | 3 | 3 | 51 | 6 | 11 | 39 |
| 13 | E. faecalis | 4 | 112 | 2 | 37 | 3 | 122 |
| | 8727 | | | | | | |
| 14 | E. faecalis | 5 | 128 | 2 | 10 | 3 | 198 |
| | 10100 | | | | | | |
| 15 | Salmonella | 5 | 4 | 51 | 8 | 18 | 211 |
| | jena | | | | | | |
| 16 | E. faecalis | 4 | 13 | 3 | 13 | 3 | 45 |
| | 8213 | | | | | | |
| 17 | E. coli 25922 | 1938 | 272 | 81 | 101 | 5 | 112 |
| 18 | E. coli 35218 | 352 | 2792 | 85 | 28 | 9 | 185 |
| 19 | E. coli 11775 | 1374 | 454 | 69 | 21 | 23 | 159 |
| 20 | MRSA 3 | 0 | 0 | 0 | 0 | 0 | 1565 |
| 21 | Salmonella | 1 | 2 | 93 | 7 | 47 | 375 |
| | dublin | | | | | | |
| 22 | L. mono 7644 | 2 | 1 | 153 | 43 | 0 | 43 |
| 23 | L. mono 7974 | 2 | 1 | 143 | 85 | 0 | 46 |
| 24 | L. mono 5214 | 2 | 1 | 22 | 39 | 0 | 39 |
| 25 | Stap aureus | 2 | 1 | 4 | 2 | 1 | 1267 |
| 26 | L. innocua | 1 | 2 | 2 | 90 | 1 | 49 |
| 27 | Micrococcus | 3 | 3 | 4 | 2 | 1 | 812 |

TABLE 13

| | | | | C | | | | |
|---|---|---|---|---|---|---|---|---|
| | | A | B | Listeria | D | E | F | |
| | | E coli | Coliform | mono | Enterococcus | Protease | Phosphatase | Code |
| 1 | MRSA 2 | − | − | − | − | − | + | F |
| 2 | MRSA 4 | − | − | − | − | − | + | F |
| 3 | MRSA 5 | − | − | − | − | − | + | F |
| 4 | L. ivanovii | − | − | + | + | − | − | CD |
| 6 | MRSA 1 | − | − | − | − | − | + | F |
| 7 | E coli 9001 | + | + | + | + | − | − | ABCD |
| 8 | E coli O157 | − | + | + | + | + | + | BCDEF |
| 9 | Citrobacter | − | + | + | + | − | − | BCD |
| 10 | E coli 8739 | + | + | + | + | − | − | ABCD |
| 11 | Finger Dry | − | − | − | + | − | + | DF |
| 12 | Finger | − | − | + | − | + | − | CE |
| | staph | | | | | | | |
| 13 | E. faecalis | − | + | − | + | − | + | BDF |
| | 8727 | | | | | | | |
| 14 | E. faecalis | − | + | − | + | − | + | BDF |
| | 10100 | | | | | | | |
| 15 | Salmonella | − | − | + | − | + | + | CEF |
| | jena | | | | | | | |
| 16 | E. faecalis | − | + | − | + | − | − | BD |
| | 8213 | | | | | | | |
| 17 | E. coli | + | + | + | + | − | + | ABCDF |
| | 25922 | | | | | | | |
| 18 | E. coli | + | + | + | + | − | + | ABCDF |
| | 35218 | | | | | | | |
| 19 | E. coli | + | + | + | + | + | + | ABCDEF |
| | 11775 | | | | | | | |
| 20 | MRSA 3 | − | − | − | − | − | + | F |
| 21 | Salmonella | − | − | + | − | + | + | CEF |
| | dublin | | | | | | | |
| 22 | L. mono 7644 | − | − | + | + | − | − | CD |
| 23 | L. mono 7974 | − | − | + | + | − | − | CD |
| 24 | L. mono 5214 | − | − | + | + | − | − | CD |
| 25 | Stap aureus | − | − | − | − | − | + | F |

1. A method of detecting target bacteria in a sample comprising;
a) providing a sample to be tested for target bacteria,
b) incubating the sample in non-selective growth medium for eight hours or less to produce a sample culture,
c) mixing some or all of the sample culture with detection reagents to produce a reaction mixture;
wherein said detection reagents comprise;
(i) a lysis reagent which disrupts bacterial cells in the sample,
(ii) a pro-luciferin molecule which is specifically converted into luciferin by said target bacteria; and,
(iii) luminescence reagents which produce a luminescent signal in the presence of luciferin;
d) incubating the reaction mixture, and;
e) measuring the luminescent signal from the reaction mixture following said incubation.

2. A method according to claim 1 wherein the production of a luminescent signal is indicative of the presence or amount of the target bacteria in the sample.

3. A method any one of the preceding claims wherein the pro-luciferin molecule is converted into luciferin by an enzyme which is characteristic of said target bacteria.

4. A method according to any one of claims 1 to 3 wherein the target microorganism is a coliform and the enzyme is beta-galactosidase.

5. A method according to claim 4 wherein the pro-luciferin molecule comprises a luciferin moiety and a beta-galactoside moiety.

6. A method according to claim 5 wherein the pro-luciferin molecule is luciferin-O-beta-galactoside.

7. A method according to any one of claims 1 to 3 wherein the target microorganism is E. coli and the enzyme is beta-glucuronidase.

8. A method according to claim 7 wherein the pro-luciferin molecule comprises a luciferin moiety and a beta-glucuronide moiety.

9. A method according to claim 8 wherein the pro-luciferin molecule is luciferin-O-beta glucuronide

10. A method according to any one of claims 1 to 3 wherein the target microorganism is a Yersinia spp, Enterococcus spp or a Listeria spp and the enzyme is beta-glucosidase.

11. A method according to claim 10 wherein the pro-luciferin molecule comprises a luciferin moiety and a beta-glucoside moiety.

12. A method according to claim 11 wherein the pro-luciferin molecule is luciferin-O-β-glucoside

13. A method according to any one of claims 1 to 3 wherein the target microorganism is a pathogenic Listeria spp and the enzyme is PiPLC (Phosphotidylinositol Phospholipase C)

14. A method according to claim 13 wherein the pro-luciferin molecule comprises a luciferin moiety and a phosphotidylinositol group.

15. A method according to claim 18 wherein the pro-luciferin molecule is luciferyl-phosphotidylinositol.

16. A method according to any one of claims 1 to 3 wherein the target microorganism is pathogenic Listeria and the enzyme is PCPLC (phosphotidylcholine phospholipase C).

17. A method according to claim 16 wherein the pro-luciferin molecule comprises a luciferin moiety and a phosphotidylcholine group.

18. A method according to claim 17 wherein the luciferin precursor is luciferyl-phosphotidylcholine.

19. A method according to any one of claims 1 to 3 wherein the target microorganism is S. aureus and the enzyme is alkaline phosphatase.

20. A method according to claim 19 wherein the pro-luciferin molecule comprises a luciferin moiety and a phosphate group.

21. A method according to claim 20 wherein the luciferin precursor is luciferin-O-phosphate.

22. A method according to any one of claims 1 to 3 wherein the target microorganism is an ESBL organism and the enzyme is β-lactamase.

23. A method according to claim 22 wherein the pro-luciferin molecule comprises a luciferin moiety and a β-lactam group.

24. A method according to claim 20 wherein the luciferin precursor is β-lactamyl-luciferin.

25. A method according to any one of claims 1 to 3 wherein the target microorganism is Enterobacter sakazakii and the enzyme is α-Glucosidase and/or β-cellobiosidase.

26. A method according to claim 25 wherein the pro-luciferin molecule comprises a luciferin moiety and an α-glucoside and/or a β-cellobioside group.

27. A method according to claim 26 wherein the luciferin precursor is luciferin-α-glucoside, luciferin-β-cellobiose or α-glucosyl-luciferin-β-cellobiose.

28. A method according to any one of claims 1 to 3 wherein the target microorganism is a Salmonella spp and the enzyme is deoxyribonuclease, α-galactosidase and/or a fatty esterase.

29. A method according to claim 28 wherein the pro-luciferin molecule comprises a luciferin moiety and a 2-deoxy-D-ribose, α-galactoside or a fatty acyl group.

30. A method according to claim 29 wherein the luciferin precursor is 2-deoxy-D-ribosyl-luciferin, luciferin-O-α-galactoside or fatty acyl-luciferin.

31. A method according to any one of claims 1 to 3 wherein the target microorganism is a cytochrome oxidase producing organism and the enzyme is cytochrome oxidase.

32. A method according to claim 31 wherein the pro-luciferin molecule comprises a luciferin moiety and an ether linked blocking group.

33. A method according to any one of claims 1 to 3 wherein the target microorganism is a protease producing bacterium and the enzyme is protease.

34. A method according to claim 33 wherein the pro-luciferin molecule comprises a luciferin moiety and a peptidyl group.

35. A method according to claim 34 wherein the luciferin precursor is aminoacyl-luciferin, di-, tri- or tetra-aminoacyl-luciferin.

36. A method according to any one of the preceding claims wherein the detection reagents comprise chemiluminescent reagents which produce a second luminescent signal in the presence of the target bacteria; or chromogenic reagents which produce a chromogenic signal in the presence of the target bacteria.

37. A method any one of the preceding claims wherein the pro-luciferin molecule is converted into luciferin by two or more enzymes, at least one of which is characteristic of said target bacteria.

38. A method any one of the preceding claims wherein said detection reagents further comprise a second pro-luciferin molecule which is specifically converted into luciferin by a second target bacteria in the sample culture; and such that the luminescence reagents which produce a second luminescent signal in the presence of the second target bacteria.

39. A method according to any one of claims 1 to 37 wherein said detection reagents further comprise a pro-coelenterazine molecule which is specifically converted into coelenterazine by a second target bacteria in the sample culture; and the luminescence reagents comprise a Renilla luciferase, such that the luminescence reagents produce a second luminescent signal in the presence of the second target bacteria.

40. A method according to any one of claims 1 to 37 wherein said detection reagents further comprise a pro-chemiluminescent or pro-fluorophore molecule which is specifically converted into a chemiluminescent molecule or fluorophore by a second target bacteria in the sample culture; such that the detection reagents produce a second luminescent signal in the presence of the second target bacteria.

41. A method according to any one of claims 1 to 37 wherein said detection reagents further comprise a pro-chromogenic molecule which is specifically converted into a chromogen by a second target bacteria in the sample culture; and such that the detection reagents produce a chromogenic signal in the presence of the second target bacteria.

42. A method of detecting target bacteria in a sample comprising;
a) providing a sample to be tested for target bacteria,
b) incubating the sample in a non-selective growth medium for eight hours or less to produce a sample culture,
wherein the non-selective growth medium further comprises;
(i) non-selective growth medium
(ii) a pro-luciferin molecule which is specifically converted into luciferin by said target bacteria,
(iii) luminescence reagents which produce a luminescent signal in the presence of luciferin; and
c) measuring the luminescent signal from the sample culture.

43. A device for detecting target bacteria in accordance with a method according to any one of claims 1 to 42, comprising;
a sample chamber for housing a sampler,
a culture medium reservoir separated from the sample chamber by a first breakable barrier; and,
a detection reagent reservoir separated from the sample chamber or the culture medium reservoir by second breakable barrier,
such that the breakage of the first breakable barrier allows growth medium in the culture medium reservoir to enter the sample chamber, and breakage of the second breakable barrier allows detection reagents from the detection reagent reservoir to enter the sample chamber and/or culture medium reservoir.

44. A kit for detecting target bacteria in accordance with a method according to any one of claims 1 to 42, comprising
a first device comprising;
a sampler, such as a dipper or swab,
a sample chamber for housing the sampler, and,
a culture medium reservoir separated from the sample chamber by a first breakable barrier; such that the breakage of the first breakable barrier allows growth medium in the culture medium reservoir to enter the sample chamber; and,
a second device comprising
a reaction chamber for accommodating a portion of sample culture from the first device; and,
a detection reagent reservoir separated from the reaction chamber by a second breakable barrier,
such that the breakage of the second breakable barrier allows detection reagents contained in the detection reagent reservoir to enter the sample chamber.

45. A device according to claim 43 or 44 wherein the culture medium reservoir contains non-specific growth medium.

46. A device according to any one of claims 43 to 45 wherein the detection reagent reservoir comprises detection reagent,
wherein said detection reagent comprises;
(i) a lysis reagent which disrupts bacterial cells in the sample
(ii) a pro-luciferin molecule which is converted into luciferin specifically by said target organism,
(iii) luminescence reagents which produce a luminescent signal in the presence of luciferin.

47. A device or kit according to any one of claims 43 to 46 wherein the sampler is a dipper or a swab

48. A device according to any one of claims 43 to 47 wherein the device comprises a buffer reservoir which is separated from the reaction chamber and/or the culture medium reservoir by a third breakable barrier, such that the breakage of the third breakable barrier allows buffer contained in the buffer chamber to enter the reaction chamber.

49. A device according to claim 48 wherein the buffer reservoir comprises buffer.

50. Use of a device according to any one of claims 43 to 49 in a method of any one of claims 1 to 42.

51. A method according to any one of claims 1 to 42 wherein steps a) to c) are performed using a device or kit according to any one of claims 43 to 49.
